(19) United States
(12) Patent Application Publication
(10) Pub. No.: US 2003/0232820 A1
(43) Pub. Date: Dec. 18, 2003

(54) Method for synthesizing oxazinones

(76) Inventors: Saul Wolfe, North Vancouver (CA); Marie-Claire Wilson, Burnaby (CA); Ming-Huei Cheng, Tainan Hsien (TW)

Correspondence Address: LAHIVE & COCKFIELD, 28 STATE STREET, BOSTON, MA, 02109, US

(21) Appl. No.: 10/231,751

(22) Filed: Aug. 29, 2002

Related U.S. Application Data
(60) Provisional application No. 60/315,602, filed on Aug. 29, 2001.

(51) Int. Cl. A61K 31/535; C07D 265/00

(52) U.S. Cl. 514/228.8; 544/63

(57) ABSTRACT

New methods and intermediates are discussed for the stereospecific synthesis of oxazinone compounds.

### RELATED APPLICATIONS

This application claims priority to U.S. Provisional Application Serial No.: 60/315,602, filed Aug. 29, 2001, entitled “Method for Synthesizing Oxazinones.” This application is also related to U.S. Pat. No. 6,399,600 B1, issued Jun. 4, 2002, entitled “Oxazinones Having Antibacterial Activity.” The entire contents of the aforementioned patent application and patent are hereby incorporated herein by reference.

### BACKGROUND OF THE INVENTION

Many antibiotics act by interfering with the biosynthesis of bacterial cell walls (Strominger et al. J. Biol. Chem. 234:3263 (1959)). The completion of bacterial cell wall synthesis is mediated by enzymes termed penicillin-binding proteins (PBPs) which cross-link different peptidoglycan chains. In particular, PBPs link the penultimate D-Ala residue of a peptidoglycan terminating in a N-acyl-D-Ala-D-Ala moiety to the terminal amino group of a lysine or diaminopimelate residue of another peptidoglycan chain, possibly through a pentaglycine bridge. Glycopeptide transpeptidase is an example of a PBP present in many bacteria.

Most known PBPs are serine peptidases, which have a conserved Ser-X-X-Lys sequence at the active site. The β-lactam family of antibiotics, whose members include penicillins and cephalosporins, inhibit PBPs by forming a covalent bond with the serine hydroxyl group to produce an acyl-enzyme. The enzyme is then unable to carry out the final step in the biosynthesis of the bacterial cell wall. As a result the wall is weakened, becomes permeable to water, and the bacterial cell swells, bursts, and dies.

The simplest kinetic description of the reaction between a bacterial enzyme (Enz) and a β-lactam antibiotic is given in Scheme 1 below:

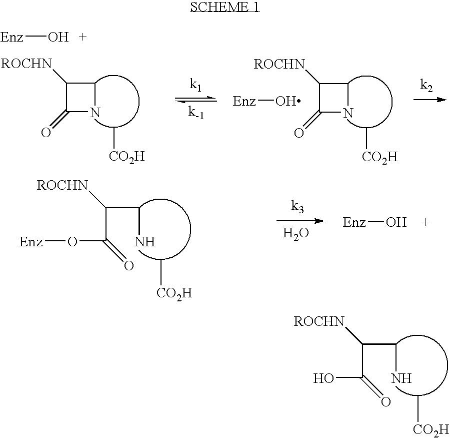

In addition to the PBP's, many bacteria also produce a second type of penicillin-recognizing enzyme, known as a β-lactamase. PBPs and β-lactamase enzymes exhibit the same kinetics as set forth in Scheme 1 above, but with different rate constants. This difference in rate constants has important consequences. In the case of PBP's, k₂>>k₃ (i.e., the formation of the acyl-enzyme is much faster than its hydrolysis). The result is that the enzyme is inhibited, and antibacterial activity may be observed. In the case of a β-lactamase, k₂≈k₃ (i.e., the formation and hydrolysis of the acyl enzyme proceed at comparable rates). These kinetics lead to regeneration of the enzyme, and inactivation of the antibiotic as a result of the net hydrolysis of the β-lactam bond in the deacylation step. The latter sequence of reactions comprises the principle mechanism of bacterial resistance to β-lactam antibiotics. Useful antibacterial activity is generally considered to require k₂/k₁≧1000M⁻¹ sec⁻¹ and k₃≦1×10⁻⁴ sec⁻¹.

Resistance to antibiotics is a problem of much current concern. Alternatives to existing antibiotics are invaluable when bacteria develop immunity to these drugs or when patients are allergic (approximately 5% of the population is allergic to penicillin). Because of the relatively low cost and relative safety of the β-lactam family of antibiotics, and because many details of their mechanism of action and the mechanism of bacterial resistance are understood, one approach to the problem of resistance is to design new classes of compounds that will complex to and react with a penicillin recognizing enzyme, and be stable to the hydrolysis step. In order to be effective, the antibacterial agent should have the ability to react irreversibly with the active site serine residue of the enzyme.

The crystal structures of β-lactamases from B. licheniformis, S. aureus and E. coli (RTEM) suggest a chemical basis for resistance to β-lactam antibiotics. Apart from the conserved Ser-X-X-Lys active site sequence, these β-lactamases have a conserved Glu166 which participates in the hydrolysis of the acyl-enzyme. It appears that the acylated hydroxyl group of the active site serine and the carboxyl group of Glu 166, together with a water molecule, are involved in the hydrolysis step. The water molecule and the carboxyl group act in concert and this interaction is the source of bacterial resistance to β-lactam antibiotics. Drug design must therefore include a process for the removal or inactivation of this water molecule.

Numerous β-lactam compounds have been developed in the past which are structural analogues of penicillin and can complex to and react with penicillin recognizing enzymes. Like penicillin, such antibiotics are presumed to be conformationally constrained analogues of an N-acyl-D-Ala-D-Ala peptidoglycan moiety, the O═C—N β-lactam bond serving as a bioisostere of the D-Ala-D-Ala peptide bond. Effective antibacterial activity also requires a properly positioned carboxyl group or equivalent and a hydrogen bonding hydroxyl or acylamino group. A computer implemented molecular modeling technique for identifying compounds which are likely to bind to the PBP active site and, thus, are likely to exhibit antibacterial activity has been developed (U.S. Pat. No. 5,552,543).

Some oxazinones having possible biological activity are known in the prior art Khomutov et al. synthesized tetrahydro-1,2-oxazin-3-one (Chem. Abs. 13754a, 1962) and 4-benzamidotetrahydro-1,2-oxazin-3-one (Chem. Abs. 58, 13944b, 1963). The latter compound is also known as N-benzoyl-cyclocanaline. According to Khomutov, cyclocanaline is known to inhibit glutamate-aspartate transaminase and exhibits activity against tuberculosis bacilli. The structure of cyclocanaline is shown in formula (A) below.

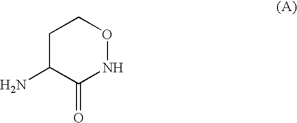

Frankel et al. reported the synthesis of DL-cyclocanaline (4-amino-tetrahydro-1,2-oxazin-3-one) hydrochloride from canaline dihydrochloride in 1969 (J. Chem. Soc. (C) 174601749, 1969) and recognized that DL-cyclocanaline is a higher homologue of the antibiotic cycloserine.

### SUMMARY OF THE INVENTION

In one embodiment, the invention pertains to a method for synthesizing an oxazinone compound of formula (I). The method includes contacting an aminooxy compound of formula (II) with a cyclizing agent under appropriate conditions, such that an oxazinone compound of formula (I) or an acceptable salt is formed. The oxazinone compound of formula (I) is:

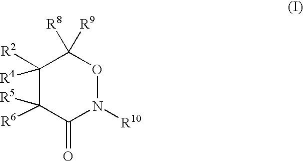

wherein

R², R⁴, R⁵, R⁶, R⁸ and R⁹ are each independently selected substituting moieties; and

R¹⁰ is hydrogen or an amino acid mimicking group. The aminooxy compound of formula (II) is:

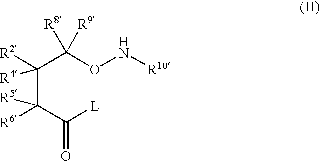

wherein:

L is a leaving group;

R^{2′}, R^{4′}, R^{5′}, R^{6′}, R^{8′} and R^{9′} are each independently selected substituting moieties; and

R^{10′} is hydrogen or an amino acid mimicking group;

In a further embodiment, the invention also pertains to aminooxy compounds of formula (II). The invention also pertains, at least in part, to all the compounds described herein as compounds and/or intermediates in the synthesis of oxazinone compounds. In an even further embodiment, the invention also includes any novel oxazinone compounds synthesized by the methods or the techniques of the invention.

In another further embodiment, the invention also pertains, at least in part, to a method for the synthesis of an oxazinone compound of formula (I) from an intermediate oxazinone. The method includes contacting an intermediate oxazinone with an N-acylating reagent under appropriate conditions. The intermediate oxazinone is of the formula (III):

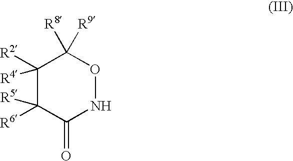

wherein:

R^{2′}, R^{4′}, R^{5′}, R^{6′}, R^{8′} and R^{9′} are each independently selected substituting moieties.

### DETAILED DESCRIPTION OF THE INVENTION

The invention pertains, at least in part, to new intermediates and synthetic methods for the stereospecific synthesis of oxazinone compounds, which are useful, for example, as antibiotics.

In one embodiment, the invention includes methods for synthesizing an oxazinone compound. The method comprises contacting an aminooxy compound with a cyclizing agent under appropriate conditions, such that an oxazinone compound or an acceptable salt is formed, wherein said aminooxy compound is of formula (II):

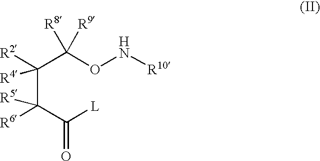

wherein:

L is a leaving group;

R^{2′}, R^{4′}, R^{5′}, R^{6′}, R^{8′} and R^{9′} are each independently selected substituting moieties; and

R^{10′} is hydrogen or an amino acid mimicking group; and wherein said oxazinone compound is of formula (I):

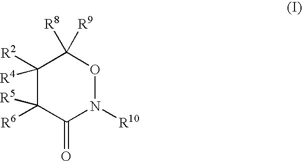

wherein

R², R⁴, R⁵, R⁶, R⁸ and R⁹ are each independently selected substituting moieties; and

R¹⁰ is hydrogen or an amino acid mimicking group.

The term “oxazinone compound” includes compounds of formula (I). The oxazinone may be formed from the precursor compound in a single synthetic step, or may be the result of a multistep synthesis. For example, the appropriate conditions may also comprise deprotecting or N-acylating reagents, such that a desired oxazinone compound is formed. Examples of oxazinone compounds which may be formed using the methods of the invention include, but are not limited to, 2R-(3-oxo-DL-4-phenylacetylamino-[1,2]oxazinan-2-yl)-propionic acid.

The term “hydroxyamine compound” includes compounds of formula (II), which can be chemically transformed through one or more chemical reactions into an oxazinone compound of formula (I). The hydroxyamine compounds of the invention may be themselves intermediates of chemical reactions and not isolated by the chemist. The term includes both isolated hydroxyamine compounds and compounds wherein the hydroxyamine compound is immediately transformed into the oxazinone compound.

The term “appropriate conditions” includes the conditions necessary for the cyclizing reagent to form the oxazinone compound. The appropriate conditions may comprise reagents, solvent, atmosphere composition, time, pressure, catalysts, and other variables known to those of skill in the art that may effect the outcome and yield of a chemical reaction. The appropriate conditions may be altered during the synthesis of an oxazinone such that a multistep synthesis can be performed. For example, for the synthesis of certain oxazinone compounds of the invention, it may be necessary to perform multistep syntheses after or before the cyclization to yield the desired oxazinone of the invention. For example, the appropriate conditions may comprise several reaction conditions (optionally with purification of the intermediates) and intermediates. Examples of other reagents which may be used to transform the aminooxy compound into a particular desired oxazinone of the invention include protecting agents, deprotecting agents, N-acylating agents, oxidizing agents, etc.

The term “cyclizing agent” includes agents and conditions which allow the oxazinone compound to be formed. Examples of cyclization agents include trimethylaluminum (Pirrung, M. C.; Chau, J, H.-L. J. Org. Chem. 1995, 60, 8084; Yamamoto, Y.; Furuta, T. Chem. Letters 1989, 797; Levin et al., Synthetic Communications, 12(13):989-993 (1982); Yamamoto et al. Chem. Lett. (1989) 797-800). When the cyclization agent is trimethylaluminum, the appropriate conditions may include, for example, 2 equivalents of AlMe₃, in a toluene solvent at reflux for 2 hours. Other appropriate conditions for cyclization using AlMe₃ include two equivalents in tetrahydrofuran at a temperature from 0° C. to room temperature over 2 hours. In another embodiment, the cyclization agent is basic reaction conditions, e.g., potassium hydroxide in methanol at reflux for two hours. In one embodiment, the yield of the desired oxazinone compound is at least about 20% or greater, about 30% or greater, about 40% or greater, about 50% or greater, about 60% or greater, about 70% or greater, about 75% or greater, about 80% or greater, about 85% or greater, or about 90% or greater. In another embodiment, the yield refers to the yield of the cyclization reaction. In another embodiment, the yield refers to the overall yield after two or more steps.

The term “leaving group” or “L” include groups which when present in the aminooxy compound result in the formation of the oxazinone compound in acceptable yields. Examples of leaving groups include alkoxy groups (e.g., methoxy) and benzhydryloxy, although other groups compatible to the chemistry of the cyclization reaction may also be substituted for the methoxy.

The term “amino acid mimicking group” includes moieties of the formula CHR¹COOR⁷, wherein R¹ is an amino acid side chain mimicking moiety and R⁷ is hydrogen, a protecting group, or a prodrug moiety. In one embodiment, the amino acid mimicking group of the oxazinone compound is capable of interacting with penicillin recognizing enzymes, such that the antibiotic activity, either directly or through decreasing the penicillin resistance of the bacteria is reduced. In another embodiment, R¹⁰ or R^{10′} is converted to an amino acid mimicking group after contacting a N-acylating reagent.

The language “amino acid side chain mimicking moiety” (“R¹”) includes moieties that are amino acid side chains or mimic amino acid side chains and which allow the oxazinone (e.g., a compound of formula II) to perform its intended function by, e.g., mimicking the structure or function of an amino acid side chain. For example, the “amino acid side chain mimicking moiety” allows the oxazinone to interact with the active site of a penicillin recognizing enzyme. Examples of amino acid side chain moieties include the side chains of natural and unnatural D- and L-amino acids. For example, the amino acid side chain mimicking moiety may be the side chain of a neutral amino acid (e.g., glycine, alanine, valine, leucine, isoleucine, phenylalanine, tryptophan, or methionine), a polar amino acid (e.g., serine, threonine, cysteine, tyrosine, asparagine, or glutamine), or a charged amino acid (e.g., aspartic acid, glutamic acid, lysine, arginine, or histidine). In one embodiment, the amino acid side chain mimicking moiety is the side chain of alanine (e.g., methyl).

In another embodiment, the amino acid side chain mimicking moiety is substituted or unsubstituted alkyl, e.g., lower alkyl. The side chain mimicking moiety may be substituted with any substituent that allows it to perform its intended function (e.g., when present in the oxazinone, it should allow the oxazinone to interact with penicillin recognizing enzyme, etc.). Examples of alkyl amino acid side chain mimicking moieties include straight chain, branched and cyclic alkyl groups. Examples of alkyl groups include, but are not limited to, methyl, ethyl, i-propyl, n-propyl, i-butyl, n-butyl, t-butyl, pentyl, cyclopentyl, cyclohexyl, or hexyl. Other examples of amino acid side chain mimicking moieties include alkenyl, alkynyl, carbonyl, aralkyl or aryl moieties. Examples of aryl moieties include substituted and unsubstituted phenyl and substituted and unsubstituted heteroaryl.

The language “protecting moiety” includes groups which can be used to protect the carboxylic acid functionality during synthesis of the oxazinone compound. Any protecting moiety known in the art and compatible with the other functionality of the oxazinones, olefinic oxazinones, and/or epoxide oxazinones may be used. Examples of protecting moieties include esters, groups known to those of skill in the art, and those described in Greene, Protective Groups in Organic Synthesis, Wiley, New York (1981), incorporated herein by reference.

The language “prodrug moiety” includes moieties which can be cleaved in vivo to yield an active drug (see, e.g., R. B. Silverman, 1992, “The Organic Chemistry of Drug Design and Drug Action”, Academic Press, Chp. 8). Prodrugs can be used to alter the biodistribution or the pharmacokinetics for a particular compound. Examples of prodrug moieties include substituted and unsubstituted, branched or unbranched lower alkyl moieties, lower alkenyl moieties, di-lower alkyl-amino lower-alkyl moieties (e.g., dimethylaminoethyl), acylamino lower alkyl moieties (e.g., acetoxymethyl), acyloxy lower alkyl moieties (e.g., pivaloyloxymethyl), aryl moieties (e.g., phenyl), aryl-lower alkyl (e.g., benzyl), substituted (e.g., with methyl, halo, or methoxy substituents) aryl and aryl-lower alkyl moieties, amides, lower-alkyl amides, di-lower alkyl amides, and hydroxy amides. Also included are groups which may not need to be removed to yield an active drug.

The term “substituting moiety” includes moieties which can be placed at any one of the R^{2′}, R^{4′}, R^{5′}, R^{6′}, R^{8′}, R^{9′}, R², R⁴, R⁵, R⁶, R⁸, R⁹ substituent of the aminooxy compound or the oxazinone compound without prohibitively detrimentally affecting the synthesis of the antibiotic. In an advantageous embodiment, each substituting moiety is selected such that the oxazinone formed may perform its intended function. Examples of substituting moieties include alkyl, hydrogen, alkenyl, alkynyl, aryl, hydroxyl, amino, protected hydroxyl, protected amino, thiol, halogen, NHCOR³, etc. and other substituents which are not detrimental to the synthesis of the oxazinone compound. Other examples of substituting moieties include carbonyl groups (e.g., R² and R⁴ or R⁵ and R⁶ taken together).

In other embodiments of the invention, the corresponding substituents of the amino hydroxy compound and the oxazinone compound may be the same or different (e.g., R² and R^{2′} are corresponding substituents, as well as R⁴ and R^{4′} and R⁵ and R^{5′}, etc.). The substituents can be converted by many reactions known in the art. For example a hydroxyl group may be converted to a carbonyl group (e.g., R² and R⁴ or R⁵ and R⁶ taken together may be a carbonyl group, for example) such that a desired oxazinone compound is synthesized.

Examples of R⁸, R^{8′}, R⁹, and R^{9′} include hydrogen and lower alkyl. Examples of R^{2′} include protected hydroxyl, hydrogen, etc. Examples of R² include protected hydroxyl, hydroxyl, hydrogen, etc. Examples of R⁴, R^{4′}, R⁶, and R^{6′} include hydrogen and lower alkyl. Examples of R^{5′} include NHCOR³, protected amino, hydrogen, etc. Examples of R⁵ include amino and NHCOR³.

In a further embodiment, the invention pertains to methods for synthesizing an oxazinone compound (wherein R² is hydroxy), by contacting an aminooxy compound (wherein R^{2′} is a protected hydroxyl group), with a cyclization agent and a deprotection agent. In a further embodiment, the method also may comprise contacting an aminooxy compound (wherein R^{2′} is hydroxyl) with a protecting agent to form the protected hydroxyl prior to cyclization to form the oxazinone.

The term “protected hydroxyl” include substituents which can be converted to a hydroxyl group upon exposure to an appropriate deprotection agent. Examples of protected hydroxyl groups include methoxymethyl (MOM). Other examples of protecting groups which can be used to protect hydroxy groups are known in the art and described in Greene, Protective Groups in Organic Synthesis, Wiley, New York (1981), incorporated herein by reference.

The term “protecting agents” include agents which can successfully protect functionality under the conditions necessary to synthesize the oxazinone compound of the invention by attaching a protecting group to the functionality of interest. Examples of protecting agents which can be used to protect hydroxyl functionality include MOM.

The term “deprotecting agents” include agents and conditions which are compatible with the functionality of the oxazinone compound and effective to remove the protecting group.

The term “derivatizing agent” includes agents which transform one functionality into another. For example, derivatizing agents can transform R⁵ from a carbamate to the desired amide.

For example, Scheme 2 below depicts a deprotection of a compound wherein R⁵ is converted from a carbamate to phenoxyacetylamino through a TFA salt intermediate. The conditions include reacting the oxazinone with TFA at a temperature of about −5° C. to room temperature to yield 98% of the salt after about an hour. The phenoxyacetyl derivative is obtained by treating the salt with phenoxyacetyl chloride in the presence of triethylamine in a dichloromethane solvent, at a temperature of about −10° C. Further examples of derivatizing agents are given in the examples.

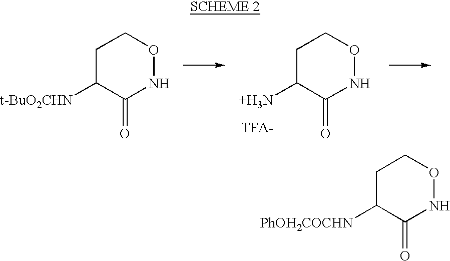

In one embodiment, R^{10′} is hydrogen. In a further embodiment, the invention pertains to methods wherein the appropriate conditions include an N-acylating agent which results in the N-acylation of either the aminooxy compound (pre cyclization) or the oxazinone compound (post cyclization) to yield an oxazinone compound wherein R¹⁰ is an amino acid mimicking group.

The term “N-acylating agent” includes agents and conditions which when reacted with oxazinone compounds (e.g., wherein R¹⁰ is, for example, hydrogen) or aminooxy compounds (e.g., wherein R^{10′} is, for example, hydrogen), such that the desired oxazinone or aminooxy compound wherein R¹⁰ or R^{10′} are an amino acid mimicking group, is formed.

In a further embodiment, the N-acylating agent and appropriate conditions are contacted with an aminooxy compound, such that an aminooxy compound wherein R^{10′} is an amino acid mimicking group is formed. In another embodiment, the N-acylating agent and appropriate conditions are contacted with the oxazinone compound (e.g., after cyclization), such that an oxazinone compound wherein R¹⁰ is an amino acid mimicking group is formed. Examples of N-acylating agents include Mitsunobu reagents, triflate reagents and reagents of formula (IV):

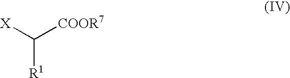

wherein X is a halogen (e.g., bromine), R¹ is an amino acid side chain mimicking moiety; and R⁷ is hydrogen, a protecting moiety, or a prodrug moiety. In a further embodiment, the appropriate conditions for N-acylating the oxazinone compound comprise Al₂O₃.

The term “triflate reagents” includes reagents of the formula (V):

CF₃SO₂O—CHR¹COOR⁷ (V)

wherein R¹ is an amino acid side chain mimicking moiety as described above and R⁷ is generally hydrogen, a protecting group, or a prodrug moiety. One example of a protecting group is benzyl. Triflate reagents can be prepared by methods known in the art. One example of a triflate reagent is the triflate of benzyl (S)-lactate. The triflate reagent may be generated in situ, and it is not necessary that the triflate reagent be isolated prior to N-acylating the compound of interest.

Appropriate conditions for this reaction include conditions which allow the reaction to take place. Examples of conditions which can be used to synthesize the triflate reagent are known in the art. Generally, the triflate reagent is formed in situ and reacted with the starting material to yield the desired N-acylated compound after an appropriate length of time.

Other N-acylating reagents include Mitsonobu reagents. The term “Mitsunobu reagents” include those chemicals and conditions which are necessary to result in the conversion of R¹⁰ or R^{10′} from hydrogen or another moiety to an amino acid mimicking group. Mitsunobu reactions are known in the art (Mitsunobu, O. Synthesis 1981, 1). Examples of Mitsunobu reagents include, but are not limited to, N-hydroxysuccinimide, diethyl azodicarboxylate and triphenylphosphine

In another embodiment, the invention includes methods for the synthesis of oxazinone compounds of formula (I), wherein R¹⁰ is an amino acid mimicking group. The method includes contacting an intermediate oxazinone with an N-acylating reagent under appropriate conditions. The intermediate oxazinone is of the formula (III):

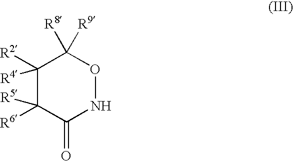

wherein:

R^{2′}, R^{4′}, R^{5′}, R^{6′}, R^{8′} and R^{9′} are each independently selected substituting moieties, and the oxazinone compound is of the formula (I):

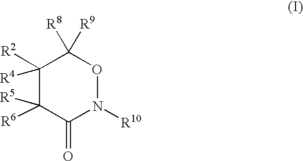

wherein:

R², R⁴, R⁵, R⁶, R⁸ and R⁹ are each independently selected substituting moieties; and

R¹⁰ is an amino acid mimicking group.

In a further embodiment, the N-acylating reagent is a reagent of formula (V), above. In a further embodiment, X is bromine and the appropriate conditions include Al₂O₃ and a salt, e.g., KF.

In one embodiment, the method further comprises synthesizing the aminooxy compound from a precursor compound, by contacting a precursor compound with reagents under appropriate conditions such that an aminooxy compound of formula (II) is synthesized. In one embodiment, the precursor compound is of formula (VI):

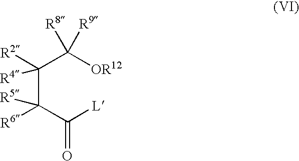

wherein:

L′ is a leaving group or a covalent bond when combined with R¹²;

R^{2″}, R^{4″}, R^{5″}, R^{6″}, R^{8″} and R^{9″} are each independently selected substituting moieties; and

R¹² is hydrogen or a covalent bond when combined with L′.

Aminooxylating agents and appropriate conditions are described in the Examples and the chemical literature (Jost, K.; Rundinger, J. Coll. Czech. Chem. Commun. 1967, 32, 2485; Manchand, et al. J. Org. Chem. 1988, 53, 5507; Bhat, B. et al. J. Org. Chem. 1996, 61, 8186).

For example, in one embodiment, examples of aminooxylating agents include those which convert R¹² from a covalent bond to hydrogen through treatment with acid. R¹² may then be converted from hydrogen to the mesylate through treatment with methanesulfonyl chloride. R¹² is then converted from the mesylate to a phthalimido group using N-hydroxyphthalimide under basic conditions. The phthalimido group can then be removed using conventional techniques and reagents such as MeNHNH₂, resulting in the formation of an aminooxy compound of formula (II). The methods of the invention also include other variations which result in the formation of an aminooxy compound of the invention.

The term “precursor compounds” include those which when treated under appropriate conditions may yield an aminooxy compound suitable for treatment with a cyclization agent under appropriate conditions such that an oxazinone compound of formula (I) is formed.

In certain embodiments, the precursor compound is a chiral compound such that the chirality can be used by a chemist to impart chirality on the desired oxazinone compound. Examples of chiral precursors which may be used include, for example, S- and L-carnitine (Bock, K. et al. Acta Chemica Scan. B37 (1983) 341-344), L-canavanine (Rosenthal, J. Agric. Food Chem. (1995) 43, 2728-2734; Miller et al. Nature, (1950) 4233:1035; Kammer et al. J Exp.Biol. (1978) 75:123-132); L-canaline (Berger, Antimicrobial Agents and Chemotherapy, 2000, 2540-2542; Rosenthal et al J. Biol. Chem. (1990) 265(2):868-873; Rosenthal et al. Comp. Biochem. Physiol. (1975) 52A:105-108; Bolkenius et al Biochem. J. (1990) 268:409-414; Rosenthal et al. Biochemical Systematics and Ecology, (1989) 17(3):203-206; Rahiala et al. Biochimica et Biophysica Acta, (1975) 227:337-343); ascorbic acid (Wei et al. J. Org. Chem. 1985, 50:3462-3467, Borek et al. J. Biol. Chem. (1938), 125:479), and cycloserine (Azarkh et al. Biokhimiya, (1960) 25(5):954-963).

In a further embodiment, the oxazinone compound of formula (I) has one of the following stereochemistries:

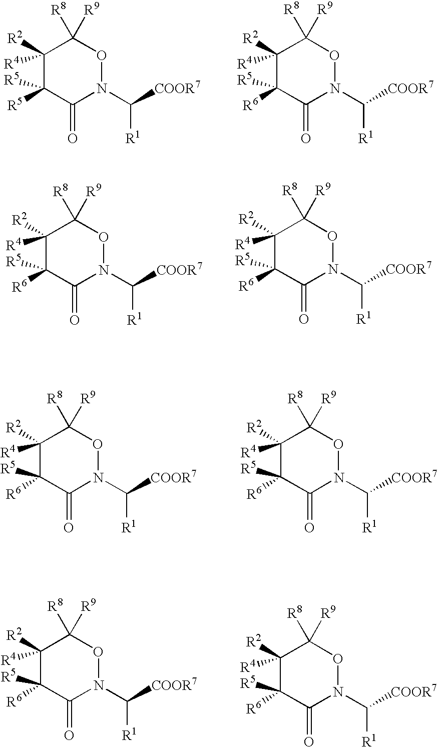

In a further embodiment, the invention pertains to aminooxy compounds of the formula (II):

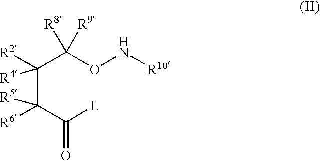

wherein:

L is a leaving group;

R^{2′}, R^{4′}, R^{5′}, R^{6′}, R^{8′} and R^{9′} are each independently selected substituting moieties; and

R^{10′} an amino acid mimicking group, or an acceptable salt thereof.

In yet another embodiment, the invention also includes methods for the synthesis of an oxazinone compound. The method may include contacting an aminooxy compound of the invention under appropriate conditions, such that an oxazinone compound is formed.

The term “alkyl” includes saturated aliphatic groups, including straight-chain alkyl groups (e.g., methyl, ethyl, propyl, butyl, pentyl, hexyl heptyl, octyl, nonyl, decyl, etc.), branched-chain alkyl groups (e.g., isopropyl, tert-butyl, isobutyl, etc.), cycloalkyl (alicyclic) groups (e.g., cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiazole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzimidazole, benzthiophene, methylenedioxyphenyl, quinoline, isoquinoline, naphthyridine, indole, benzofuran, purine, benzofuran, diazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including, e.g., alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. The term “substituted acyl” includes acyl groups where one or more of the hydrogen atoms are replaced by, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes structures wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthylcarboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkylamino” includes groups and compounds wherein the nitrogen is bound to at least one additional alkyl group. The term “dialkylamino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkylaminoalkyl” refers to an alkyl, alkenyl, or alkynyl group bound to a nitrogen atom which is also bound to an alkyl group.

The term “amide” or “aminocarboxy” includes compounds or moieties which contain a nitrogen atom which is bound to the carbon of a carbonyl or a thiocarbonyl group. The term includes “alkylaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarbony groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkylthioalkyls, alkylthioalkenyls, and alkylthioalkynyls. The term “alkylthioalkyls” include compounds with an alkyl, alkenyl, or alkynyl group bonded to a sulfur atom which is bonded to an alkyl group. Similarly, the term “alkylthioalkenyls” and alkylthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminocarbonyl, arylalkylaminocarbonyl, alkenylaminocarbonyl, alkylcarbonyl, arylcarbonyl, arylalkylcarbonyl, alkenylcarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

It will be noted that the structure of some of the compounds of this invention includes stereogenic carbon atoms. It is to be understood accordingly that the isomers arising from such asymmetry (e.g, all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

The invention also pertains, at least in part, to compounds, precursors, and intermediates described herein, as both chemical compositions and, if applicable, as pharmaceutical compositions. The invention also pertains to therapeutic methods for using these compositions, e.g., as antibiotics.

The invention is further illustrated by the following examples, which should not be construed as further limiting.

### EXEMPLIFICATION OF THE INVENTION

Experimental

All reactions were performed under dry nitrogen using oven-dried (140° C., 24 h) glassware, which was allowed to cool in a desiccator under vacuum. Solvents were distilled prior to use and dried according to standard literature procedures (Perrin, D. D.; Armarego, W. L. F. Purification of Laboratory Chemicals. Pergamon Press, 3rd Edition, 1988). Both ¹H and ¹³C nuclear magnetic resonance (NMR) spectra were obtained on a Bruker Model AMX 400 Spectrometer operating at 400.1 MHz. Chemical shifts (δ) are reported in parts per million (ppm) downfield from internal tetramethylsilane (TMS) in an appropriate deuterated solvent. Infrared (IR) spectra were obtained on a Perkin-Elmer 599B spectrometer (neat film, 1-2% KBr pellet or 1% solution). Low-resolution mass spectra, refer to direct inlet electron impact (EI) measurements or chemical ionization (CI) measurements, were recorded on a Hewlett-Packard 5985 GC/MS/IS system. Elemental analyses were performed on a Carlo Erba model 1106 elemental analyzer. Melting points (m.p.) were obtained on a Fisher-Johns apparatus, and are uncorrected. Preparative layer chromatography (PLC) was carried out on precoated Merck Silica Gel 60 F-254 plates with aluminum backing. Spots were observed under short-wavelength ultraviolet light, and were visualized with a solution of 1% ceric sulfate, or 2% molybdic acid in 10% sulfuric acid. Flash column chromatography was carried out on Merck Silica Gel 60 (230-400 Mesh).

### Example 1

### Synthesis of 4-Phenylacetylamino-[1,2]Oxazinan-3-one

α-Amino-γ-Butyrolactone Hydrobromide (A)

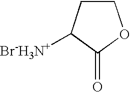

A solution of DL-homoserine (0.31 g, 2.6 mmoles) in 2.4 M hydrobromic acid (5 mL, 12 mmoles, 4.6 eq) was refluxed for 3 h and then stirred overnight. Removal of the solvent afforded a white solid, which was dissolved in ethanol and cooled to give the product as a solid which was collected by filtration and washed with cold ethanol to give 0.384 g (81%) of the product (A), m.p. 117-119° C. Calcd. for C₄H₈NO₂Br: C, 26.40; H, 4.44; N, 7.70. Found: C, 26.35; H, 4.25; N, 7.51.

Phenylacetyl Chloride

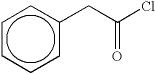

A solution of phenylacetic acid (1.55 g, 11.4 mmoles) in thionyl chloride (5 mL, 8.16 g, 70.0 mmoles) was refluxed for 1 h, cooled to room temperature, concentrated and distilled to give 1.59 g (90%) of the acid chloride, b.p. 45-46°/1 torr. ¹Hmr (CDCl₃, δ): 7.41 (5H, m, Ar), 4.19 (2H, s, CH₂). IR (neat): 3034, 1800 cm⁻¹.

α-Phenylacetamido-γ-Butyrolactone

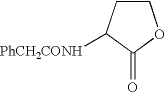

Triethylamine (7.7 mL, 5.6 g, 55.0 mmoles) was added slowly to a suspension of α-amino-γ-butyrolactone hydrobromide (A) (5.0 g, 27.5 mmoles) in dichloromethane (50 mL). The mixture was stirred for 10 min, cooled to −5° C. and treated, during 30 min, with a solution of phenylacetyl chloride (3.3 mL, 3.83 g, 24.8 mmoles) in dichloromethane (20 mL). The mixture was allowed to warm to room temperature, stirred for 3 h, and then washed successively with water (25 mL), N hydrochloric acid (15 mL), water (15 mL) and 1:1 water:saturated sodium bicarbonate (20 mL), dried over anhydrous magnesium sulfate and evaporated to give a white solid (5.14 g, 95%), m.p. 124-126° C. ¹Hmr (CDCl₃, δ): 7.33 (5H, m, Ar), 5.97 (1H, br s, NH), 4.50 (1H, m, CHγ), 4.43 (1H, t, 9.1 Hz, CHα), 4.25 (1H, m, CHγ), 3.63 (2H, s, PhCH₂), 2.79 (1H, m, CHβ), 2.08 (1H, m, CHβ). ¹³Cmr (CDCl₃, δ): 174.98, 171.49 134.06, 129.39, 129.13, 127.59, 65.95, 49.37, 43.31, 30.32. IR (KBr): 3305, 1779, 1652 cm⁻¹. Mass spectrum (CI, m/z): 220 (M+1). Calcd. for C₁₂H₁₃NO₃: C, 65.73; H, 5.99; N, 6.39. Found: C, 65.69; H, 5.97; N, 6.12.

N-Phenylacetyl Homoserine

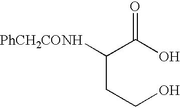

A solution of potassium hydroxide (4.51 g, 68.8 mmoles) in methanol (20 mL) was added in portions to a suspension of α-phenylacetamido-γ-butyrolactone (10.1 g, 45.9 mmoles) in dry methanol (100 mL). The mixture was stirred at room temperature for 24 h and Amberlyst 15 (H⁺) (18.5 g, 82.5 mmoles), presoaked in methanol, was added. The mixture was filtered and the filtrate was evaporated to an oil (10.9 g, 96%), which was used directly in the next step.

Methyl N-Phenylacetyl Homoserinate (B)

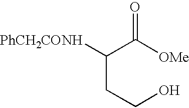

A solution of N-phenylacetyl homoserine (3.19 g, 13.5 mmoles) in dimethylformamide (30 mL) was treated, in portions, with a solution of triethylamine (1.88 mL, 1.36 g, 13.5 mmoles) in dimethylformamide (10 mL). Then iodomethane (1.68 mL, 3.82 g, 26.9 mmoles) was added in one portion and stirring was continued overnight. Most of the solvent was removed under reduced pressure and the residue was diluted with ethyl acetate (100 mL) and water (20 mL). The aqueous layer was washed with ethyl acetate (2×25 mL) and the combined organic layers were washed successively with 5% citric acid (2×15 mL), water (15 mL), saturated sodium sulfite (15 mL), saturated sodium bicarbonate (2×15 mL) and saturated sodium chloride (2×15 mL), dried over anhydrous magnesium sulfate and evaporated to give a pale yellow oil (2.02 g, 60%). ¹Hmr (CDCl₃, δ): 7.32 (5H, m, Ar), 6.38 (1H,br d, 6.8 Hz, NH), 4.73 (1H, m, CHα), 3.73 (3H, s, CH₃), 3.64 (1H, m, CHγ), 3.63 (2H, s, PhCH₂),3.49 (1H, m, CHγ), 2.33 (1H, br s, OH), 2.13 (1H, m, CHβ), 1.56 (1H, m, CHβ). ¹³Cmr (CDCl₃, δ): 172.94, 172.15, 134.21, 129.32, 129.09, 127.56, 58.09, 52.64, 49.66, 43.45, 35.63. IR (CH₂Cl₂): 3297, 1743, 1658 cm⁻¹. Mass spectrum (CI, m/z): 252 (M+1).

Methyl N-Phenylacetyl-O-Methanesulfonyl Homoserinate (C)

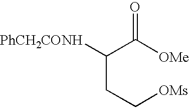

A solution of methyl N-phenylacetyl homoserine (B) (1.90 g, 7.56 mmoles) and triethylamine (1.26 mL, 0.91 g, 9.07 mmoles), in dichloromethane (30 mL), was cooled to −15 to −10° C. and methanesulfonyl chloride (0.67 mL, 1.00 g, 8.70 mmoles) was added dropwise. The mixture was allowed to warm to 0° C. and was stirred at that temperature for 1 h. Cold 10% potassium bisulfate (15 mL) was added and the aqueous layer was separated and washed with dichloromethane (2×15 mL). The combined organic layers were washed with sodium bicarbonate (2×25 mL), dried over anhydrous magnesium sulfate, and evaporated to give a yellow oil (2.23 g, 90%). ¹Hmr (CDCl₃, δ): 7.38 (5H, m, Ar), 6.19 (1H, br d, 7.1 Hz, NH), 4.70 (1H, m, CHα), 4.17 (2H, t, 6.2 Hz, CH₂γ), 3.76 (3H, s, COCH₃), 3.64 (2H, s, PhCH₂), 2.90 (3H, s, SO₂CH₃), 2.34 (1H, m, CHβ), 2.12 (1H, m, CHβ). ¹³Cmr (CDCl₃, δ): 171.73, 171.02, 134.36, 129.34, 129.12, 127.55, 65.68, 52.77, 49.34, 43.63, 37.16, 31.46. Mass spectrum (CI, m/z): 330 (M+1).

Methyl α-Phenylacetylamino-γ-Phthalimidooxybutyrate (D)

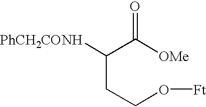

Triethylamine (0.95 mL, 0.69 g, 6.78 mmoles) was added slowly to a solution of N-hydroxyphthalimide (1.11 g, 6.78 mmoles) in acetonitrile (15 mL). The solution was stirred for 20 min, cooled to 10 to 15° C., and a solution of methyl N-phenylacetyl-O-methanesulfonyl homoserinate (C) (2.23 g, 6.78 mmoles) in acetonitrile (5 mL) was added dropwise. The reaction mixture was allowed to warm to room temperature and stirring was continued for 24 h. The solvent was removed and the residue was dissolved in ethyl acetate (50 mL). This solution was washed successively with water (2×15 mL), saturated sodium bicarbonate (8×15 mL), water (15 mL), 5% citric acid (15 mL), water (15 mL) and saturated sodium chloride (15 mL), dried over anhydrous magnesium sulfate, and evaporated. The resulting solid was recrystallized from hot ethanol to give 2.25 g (84%), m.p. 138-139° C. ¹Hmr (CDCl₃, δ): 7.81 (4H, m, phthalimido ring), 7.24 (5H, m, Ph), 6.15 (1H, br s, NH), 4.87 (1H, m, CHα), 4.21 (2H, t, 5.8 Hz, CH₂γ), 3.73 (3H, s, CH₃), 3.67 (2H, s, PhCH₂), 2.30 (2H, m, CH₂β). IR (KBr): 3348, 1792, 1735, 1664 cm⁻¹. Mass spectrum (CI, m/z): 397 (M+1). Calcd. for C₂₁H₂₀N₂O₆: C, 63.62; H, 5.10; N, 7.07. Found: C, 63.97; H, 5.19; N, 7.12.

Methyl 2-Phenylacetylamino-4-Aminooxybutyrate (E)

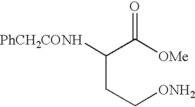

Methylhydrazine (0.20 mL, 0.174 g, 3.78 mmoles) was added dropwise at −10° C. to a solution of methyl α-phenylacetylamino-γ-phthalimidooxybutanoate (D) (1.0 g, 2.52 mmoles) in dichloromethane (40 mL). The cooling bath was removed and stirring was continued for 3 h. The mixture was then filtered and the filtrate was evaporated. The residue, in ethyl acetate (30 mL), was washed with 1:1 saturated sodium chloride:saturated sodium bicarbonate (6 mL). The aqueous layer was extracted with ethyl acetate (30 mL) and the combined organic extract was dried over anhydrous magnesium sulfate and evaporated to give a pale yellow oil (0.664 g, 99%). ¹Hmr (CDCl₃, δ): 7.35 (5H, m, Ph), 6.50 (1H, br s, NH), 4.68 (1H, m, CHα), 3.75 (3H, s, COCH₃), 3.65 (2H, t, 5.8 Hz, CH₂γ), 3.65 (2H, s, PhCH₂), 2.03 (2H, m, CH₂β). Mass spectrum (CI, m/z): 267 (M+1).

4-Phenylacetylamino-[1,2]Oxazinan-3-one (F)

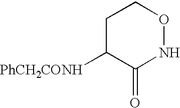

A 2.0 M solution of trimethylaluminum in hexanes (1.13 mL, 2.25 mmoles) was added dropwise, at 0° C., to the canaline derivative (E) (0.300 g, 1.13 mmoles) in toluene (20 mL). The ice-bath was removed and stirring was continued for 4 h. The reaction mixture was then treated dropwise with water (1 mL). After an additional 15 min, the solvent was removed under reduced pressure to near-dryness, 1:4 dichloromethane:tetrahydrofuran (150 mL) was added, and the mixture was filtered through Celite. The filtrate was concentrated and the residue was purified by short column chromatography on silica gel with a gradient solvent system, hexanes to ethyl acetate, to give a white solid (91.2 mg, 35%), m.p. 164-165° C. ¹Hmr (CDCl₃, δ): 8.17 (1H, br s, ONH), 7.32 (5H, m, Ar), 6.41 (1H, br s, NH), 4.72 (1H, m, CHα), 4.23 (1H, m, CHγ), 4.04 (1H, m, CHγ), 3.63 (2H, s, PhCH₂), 2.98 (1H, m, CHβ), 1.63 (1H, m, CHβ). ¹³Cmr (CDCl₃, δ): 173.52, 171.08, 134.45, 129.30, 128.96, 127.37, 69.84, 47.02, 43.56, 28.99. IR (KBr): 3307, 1694, 1660 cm⁻¹. Mass spectrum (CI, m/z): 235 (M+1). Calcd. for C₁₂H₁₄N₂O₃: C, 61.52; H, 6.04; N, 11.96. Found: C, 61.32; H, 6.04; N, 11.92.

### Example 2

### Synthesis of 4-Phenoxyacetylamino-[1,2]Oxazinan-3-one

Triethylamine Salt of N-BOC-Homoserine

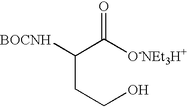

A solution of homoserine (5.00 g, 42.0 mmoles) in 50% aqueous acetone (50 mL), was treated with triethylamine (8.8 mL, 6.39 g, 63.0 mmoles) and BOC anhydride (10.1 g, 46.2 mmoles), and stirred overnight at room temperature. Removal of the solvent gave the salt (13.5 g, 100%). ¹Hmr (D₂O, δ): 4.02 (1H, m, CHα), 3.64 (2H, m, CH₂γ), 3.18 (2H, q, 7.3 Hz, CH₂CH₃) 1.98 (1H, m, CHβ), 1.82 (1H, m, CHβ), 1.41 (9H, s, C(CH₃)₃), 1.26 (3H, t, 7.3 Hz, CH₃). ¹³Cmr (D₂O, δ): 181.68, 160.33, 83.76 61.09, 55.48, 49.30, 36.57, 30.28, 10.85. IR (CH₂Cl₂): 3413, 1709, 1693 cm⁻¹. Mass spectrum (CI, m/z): 164 (M+1-BOC).

Methyl N-BOC-Homoserinate (G)

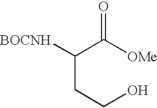

Method A

A solution of the triethylamine salt of N-BOC-homoserine (7.49 g, 23.4 mmoles) in dimethylformamide (50 mL) was cooled to 0-10° C., stirred, iodomethane (1.6 mL, 3.65 g, 25.7 mmoles) was added, the cooling bath was removed, and stirring was continued overnight. Most of the solvent was removed under reduced pressure and the residue was diluted with ethyl acetate (80 mL) and washed with water (20 mL). The aqueous extract was washed with ethyl acetate (2×25 ml), and the combined organic extracts were washed successively with 5% citric acid (2×15 ml), water (15 mL), saturated sodium sulfite (15 mL), saturated sodium bicarbonate (2×15 ml) and saturated sodium chloride (2×15 ml), dried over anhydrous magnesium sulfate and evaporated to give a colorless oil (5.15 g, 94%). ¹Hmr (CDCl₃, δ): 5.43 (1H, br s, NH), 4.52 (1H, m, CHα), 3.80 (3H, s, CH₃O), 3.73 (2H, m , CH₂γ), 3.26 (1H, br s, OH), 2.11 (1H, m, CHβ), 1.64 (1H, m, CHβ), 1.49 (9H, s, C(CH₃)₃). ¹³Cmr (CDCl₃, δ): 173.62, 156.80, 80.16, 58.65, 52.50, 51.04, 36.18, 28.66. Mass spectrum (CI, m/z): 234 (M+1).

Method B

To a solution of N-BOC-homoserine (7.00 g, 31.9 mmoles) in dimethylformamide (40 mL) was added, during 30 min, a solution of triethylamine (4.50 mL, 3.27 g, 32.2 mmoles) in dimethylformamide (15 mL). Iodomethane (4.00 mL, 9.11 g, 64.2 mmoles) was then added in one portion and the reaction mixture was stirred overnight, and then concentrated under reduced pressure to remove most of the solvent. The residue was diluted with ethyl acetate (100 mL) and water (20 mL), the aqueous layer was washed with ethyl acetate (2×25 ml), and the combined organic layers were washed successively with 5% citric acid (2×15 ml), water (15 mL), saturated sodium sulfite (15 mL), saturated sodium bicarbonate (2×15 ml) and saturated sodium chloride (2×15 ml), dried over anhydrous magnesium sulfate and evaporated to give a colourless oil (5.79 g, 78%), identical with the product of Method A.

Methyl 2-tert-Butoxycarbonylamino-4-Methanesulfonylooxybutyrate (H)

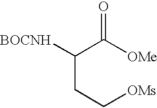

Methanesulfonyl chloride (2.14 mL, 3.16 g, 27.6 mmoles) was added dropwise at −15 to −10° C. to a solution of methyl N-BOC-homoserinate (G) (5.6 g, 24.0 mmoles) and triethylamine (4.0 mL, 2.90 g, 28.8 mmoles) in dichloromethane (80 mL). The reaction mixture was allowed to warm to 0° C. and was stirred at that temperature for 1 h. Cold 10% potassium bisulfate (25 mL) was added, and the aqueous layer was separated and washed with dichloromethane (2×25 ml). The combined organic layers were washed with sodium bicarbonate (2×35 ml), dried over anhydrous magnesium sulfate, and evaporated to give a pale yellow oil (7.25 g, 97%). ¹Hmr (CDCl₃, δ): 5.25 (1H, br s, NH), 4.75 (1H, m, CHα), 4.35 (2H, m, CH₂γ), 3.82 (3H, s, CH₃O), 3.07 (3H, s, SO₂CH₃), 2.36 (1H, m, CHβ), 2.15 (1H, m, CHβ), 1.49 (9H, s, C(CH₃)₃).

Methyl N-BOC-α-Amino-γ-Phthalimidooxybutyrate (I)

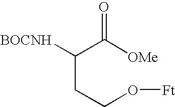

1,8-Diazabicyclo[5.4.0]undec-7-ene (3.2 mL, 3.18 g, 20.9 mmoles) was added dropwise to a solution of N-hydroxyphthalimide (3.40 g, 20.9 mmoles) in dimethylformamide (30 mL). The solution was stirred for 30 min, cooled to 10-15° C. and a solution of methyl 2-tert-butoxycarbonylamino-4-methanesulfonyloxybutyrate (H) (6.5 g, 20.9 mmoles) in dimethylformamide (10 mL) was added dropwise. The mixture was then allowed to warm to room temperature and stirred for 48 h. Approximately 30 mL of solvent were removed under reduced pressure and ethyl acetate (100 mL) and water (30 mL) were added. The layers were separated and the aqueous layer was washed with ethyl acetate (2×50 mL ). The combined organic layers were washed successively with saturated sodium bicarbonate (5×30 mL), water (30 mL), 5% citric acid (30 mL), water (30 mL) and saturated sodium chloride (30 mL), dried over anhydrous magnesium sulfate, and evaporated to yield 8.58 g of a solid. Recrystallization from hot ethanol gave white crystals, 5.07 g (64%), m.p. 135-136° C. ¹Hmr (CDCl₃, δ): 7.81 (4H, m, phthalimido ring), 5.68 (1H, d, 7.6 Hz, NH), 4.55 (1H, m, CHα), 4.31 (2H, t, 6.1 Hz, CH₂γ), 3.76 (3H, s, CH₃), 2.30 (2H, m, CH₂β), 1.45 (9H, s, C(CH₃)₃). IR (KBr): 3365, 1746, 1725, 1680 cm⁻¹. Mass spectrum (CI, m/z): 379 (M+1). Calcd. for C₁₈H₂₂N₂O₇: C, 57.13; H, 5.87; N, 7.40. Found: C, 57.21; H, 5.93; N, 7.33.

Methyl N-BOC-α-Amino-γ-Aminooxybutyrate (J)

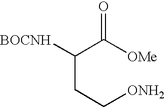

A solution of methyl N-BOC-α-amino-γ-phthalimidooxybutyrate (I) (0.50 g, 1.32 mmoles) in dichloromethane (20 mL) was cooled to −10° C. and methylhydrazine (0.10 mL, 91.1 mg, 1.98 mmoles) was added dropwise, with stirring. Stirring was continued for 2 h at −10 to 0° C. and the mixture was then filtered. The filtrate was concentrated and the residue, in ethyl acetate (15 mL), was washed with 1:1 saturated sodium chloride:saturated sodium bicarbonate (10 mL). The aqueous layer was washed with ethyl acetate (15 mL) and the combined organic layers were dried over anhydrous magnesium sulfate and evaporated to give a white solid (0.325 g, 100%). ¹Hmr (CDCl₃, δ): 5.62 (2H, br s, ONH₂), 5.29 (1H, d, 6.0 Hz, NH), 4.41 (1H, m, CHα), 3.75 (3H, s, CH₃), 3.75 (2H, t, 6.2 Hz, CH₂γ), 2.07 (2H, m, CH₂β), 1.45 (9H, s, C(CH₃)₃). ¹³Cmr (CDCl₃, δ): IR (KBr): 3336, 1738, 1698 cm⁻¹. Mass spectrum (CI, m/z): 249 (M+1).). Calcd. for C₁₀H₂₀N₂O₅: C, 48.37; H, 8.13; N, 11.28. Found: C, 48.77; H, 7.96; N, 11.36.

4-tert-Butoxycarbonylamino-[1,2]Oxazinan-3-one (K)

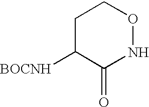

A solution of methyl N-BOC-α-amino-γ-aminooxybutyrate (J) (0.150 g, 0.607 mmole) in tetrahydrofuran (15 mL) was cooled to 0° C. and a 2.0 M solution of trimethylaluminum in heptane (0.60 mL, 1.21 mmoles) was added dropwise. The reaction mixture was allowed to warm to room temperature and was stirred for 4 h. Water (2 mL) was added dropwise and, after 15 min, the solution was concentrated to near dryness, diluted with 1:4 dichloromethane:tetrahydrofuran (50 mL) and filtered through Celite. The filtrate was concentrated to give a white solid (128 mg, 98%). m.p. 119-120° C. ¹Hmr (CDCl₃, δ): 8.65 (1H, br s, ONH), 5.43 (1H, br s, NH), 4.54 (1H, m, CHα), 4.21 (1H, m, CHγ), 4.08 (1H, m, CHγ), 2.88 (1H, m, CHβ), 1.74 (1H, m, CHβ), 1.46 (9H, s, C(CH₃)₃). ¹³Cmr (CDCl₃, δ): 174.11, 155.35, 80.03, 69.77, 47.78, 29.74, 28.09. IR (KBr): 3364, 1696, 1679, 1528 cm⁻¹. Mass spectrum (CI, m/z): 217 (M+1). Calcd. for C₉H₁₆N₂O₄: C, 49.98; H, 7.47; N, 12.96. Found: C, 50.17; H, 7.47; N, 12.74.

TFA Salt of Cyclocanaline

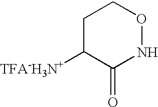

4-tert-Butoxycarbonylamino-[1,2]oxazin-3-one (K) (50.9 mg, 0.235 mmoles) was added, with stirring, to a cooled (0 to −5° C.) solution of trifluoroacetic acid (0.75 mL). The mixture was allowed to warm to room temperature and was stirred for 1 h. The solvent was then removed under reduced pressure and the residue was triturated with ether to give a white solid (53.0 mg, 98%). ¹Hmr (D₂O, δ): 4.29 (1H, m, CHγ), 4.09 (1H, m, CHγ), 4.08 (1H, m, CHα), 2.77 (1H, m, CHβ), 2.06 (1H, m, CHβ).

4-Phenoxyacetylamino-[1,2]Oxazinan-3-one (L)

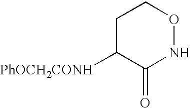

Triethylamine (0.2 mL, 0.145 g, 1.43 mmoles) was added slowly to a suspension of the trifluoroacetic acid salt of cyclocanaline (0.150 g, 0.63 mmole) in dichloromethane (5 mL), the mixture was cooled to −10° C., and a solution of phenoxyacetyl chloride (0.079 mL, 97.2 mg, 0.57 mmole) in dichloromethane (2 mL) was added dropwise. The reaction mixture was allowed to warm to room temperature and was stirred overnight and then washed successively with water (2 mL), 5% citric acid (2 mL), water (2 mL) and 1:1 saturated sodium chloride:saturated sodium bicarbonate (2 mL), dried over anhydrous magnesium sulfate, and evaporated. The residue was purified by short column chromatography on silica gel, using hexanes to ethyl acetate gradient solvent system, to give a white solid (29.8 mg, 38%). ¹Hmr (CDCl₃, δ): 7.53 (1H, br s, NH), 7.15 (5H, m, Ar), 4.84 (1H, m, CHα), 4.55 (2H, s, PhOCH₂), 4.28 (1H, m, CHγ), 4.11 (1H, m, CHγ), 3.03 (1H, m, CHβ), 1.78 (1H, m, CHβ). ¹³Cmr (CDCl₃, δ): 173.06, 168.47, 157.20, 129.75, 122.17, 114.79, 69.89, 67.29, 46.66, 28.94. IR (KBr): 3314, 1686, 1655 cm⁻¹. Mass spectrum (CI, m/z): 251 (M+1).

### Example 3

### Synthesis of 2R-(3-Oxo-[1,2]Oxazinan-2-yl)-Propionic Acid

Methyl γ-Bromobutyrate (M)

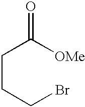

A solution of γ-butyrolactone (25 mL, 28.0 g, 0.325 mole) in a 45% solution of hydrogen bromide in acetic acid (75 mL) was heated for 4 h at 75° C., cooled to room temperature, treated with methanol (150 mL), and stirred overnight. Evaporation of the solvent gave a dark oil, which was dissolved in ethyl acetate (150 mL) and washed successively with saturated sodium bicarbonate (2×150 mL), saturated sodium chloride (150 mL), dried over anhydrous magnesium sulfate, and evaporated to yield a clear oil (48.8 g, 86%). ¹Hmr (CDCl₃, δ): 3.68 (3H, s, OCH₃), 3.44 (2H, t, 6.4 Hz, CH₂γ), 2.51 (2H, t, 7.2 Hz, CH₂α), 2.17 (2H, quintet, 6.8 Hz, CH₂β). ¹³Cmr (CDCl₃, δ): 172.96, 51.70, 32.66, 32.19, 27.70. IR (neat): 1738 cm⁻¹. Mass spectrum (CI, m/z): 181 (M+1), 183 (M+3). Calcd. for C₅H₉O₂Br: C, 33.17; H, 5.02. Found: C, 32.62; H, 4.85.

Methyl γ-Phthalimidooxybutyrate (N)

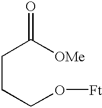

A mixture of methyl γ-bromobutyrate (M) (14.7 g, 81.5 mmoles), N-hyroxyphthalimide (13.3 g, 81.5 mmoles) and triethylamine (22.7 mL, 16.5 g, 0.163 mole), in acetonitrile (110 mL) was refluxed for 3 h. The insoluble solid was removed by filtration and the filtrate was evaporated. The residue was diluted with ethyl acetate (100 mL) and this solution was washed successively with water (3×100 mL) and saturated sodium chloride (100 mL), dried over anhydrous magnesium sulfate and evaporated. The solid was recrystallized from hot ethanol to give 16.8 g, (78%), m.p. 79-81° C. ¹Hmr (CDCl₃, δ): 7.79 (4H, m, phthalimido ring), 4.26 (2H, t, 6.1 Hz, CH₂γ), 3.70 (3H, s, OCH₃), 2.66 (2H, t, 7.3 Hz, CH₂α), 2.09 (2H, quintet, 6.7 Hz, CH₂β). ¹³Cmr (CDCl₃, δ): 174.50, 163.63, 134.43, 128.94, 123.48, 78.20, 51.50, 33.85, 25.14. IR (KBr): 1735, 1727 cm⁻¹. Mass spectrum (CI, m/z): 264 (M+1). Calcd. for C₁₃H₁₃NO₅: C, 59.30; H, 4.99; N, 5.32. Found: C, 59.17; H, 4.97; N, 5.49.

Methyl γ-Aminooxybutyrate (O)

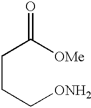

A solution of methyl γ-phthalimidooxybutyrate (N) (2.75 g, 10.45 mmoles) in dichloromethane (50 mL) was cooled to −10° C. and treated dropwise, with stirring, with methylhydrazine (0.83 mL, 0.72 g, 15.7 mmoles). Stirring was continued for 1.5 h at −10 to 0° C. and the mixture was then filtered. The filtrate was concentrated and the residue, in ethyl acetate (50 mL), was washed with 1:1 saturated sodium chloride:saturated sodium bicarbonate (22 mL). The aqueous layer was washed with ethyl acetate (50 mL) and the combined organic extracts were dried over anhydrous magnesium sulfate and evaporated to give a yellow oil (1.39 g, 100%). ¹Hmr (CDCl₃, δ): 5.25 (2H, br s, NH₂), 3.68 (2H, t, 6.2 Hz, CH₂γ), 3.68 (3H, s, OCH₃), 2.38 (2H, t, 7.3 Hz, CH₂α), 1.92 (2H, quintet, 6.7 Hz, CH₂β). ¹³Cmr (CDCl₃, δ): 173.95, 74.78, 51.58, 30.71, 23.65. IR (neat): 3322, 1734 cm⁻¹. Mass spectrum (CI, m/z): 134 (M+1). Calcd. for C₅H₁₁NO₃: C, 45.09; H, 8.34; N, 10.52. Found: C, 45.02; H, 8.36; N, 10.49.

Benzyl (S)-Lactate

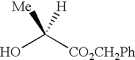

1,8-Diazabicyclo[5.4.0]undec-7-ene (18 mL, 18.3 g, 0.12 mole) was added slowly, with stirring, to a solution of 85% (S)-lactic acid (12.7 g, 0.12 mole) in methanol (50 mL). The solvent was removed under reduced pressure at 70-80° C., and the resulting oil, in dimethylformamide (50 mL), was cooled to 15° C. Benzyl bromide (11.9 mL, 17.1 g, 0.10 mole) was added dropwise and the reaction mixture was stirred at room temperature for 30 h. After removal of approximately 40 mL of solvent by vacuum distillation, ethyl acetate (100 mL) was added, followed by water (30 mL). The aqueous layer was washed with ethyl acetate (2×30 mL) and the combined organic extracts were washed successively with water (30 mL), 5% citric acid (30 mL), water (30 mL), saturated sodium bicarbonate (3×30 mL) and saturated sodium chloride (2×30 mL), dried over anhydrous magnesium sulfate, and evaporated. The crude product was distilled, to give a colourless oil (13.8 g, 76%), b.p. 119-123°/1 torr. ¹Hmr (CDCl₃, δ): 7.37 (5H, m, Ar), 5.21 (2H, s, PhCH₂), 4.32 (1H, q, 6.9 Hz, CH), 2.38 (1H, br s, OH), 1.43 (3H, d, 6.9 Hz, CH₃).). ¹³Cmr (CDCl₃, δ): 175.52, 135.23, 128.66, 128.54, 128.22, 67.31, 66.84, 20.37. IR (neat): 3483, 1738 cm⁻¹. Mass spectrum (CI, m/z): 181 (M+1).). Calcd. for C₁₀H₁₂O₃: C, 66.64; H, 6.73. Found: C, 66.30; H, 6.75.

4-[N-(1-Benzyloxycarbonyl-ethyl)aminooxy]-butyric Acid Methyl Ester (P)

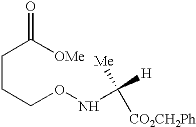

A solution of 2,6-lutidine (0.35 mL, 0.32 g, 3.0 mmoles) in dichloromethane (3 mL) was added at −78° C. to a solution of benzyl (S)-lactate (0.271 g, 1.5 mmoles) in dichloromethane (15 mL). The solution was stirred for 10 min, trifluoromethanesulfonic anhydride (0.25 mL, 0.42 g, 1.5 mmoles) was added dropwise and stirring was continued for 30 min at −78 to −50° C. A solution of methyl γ-aminooxybutrate (O) (0.170 g, 1.28 mmoles) and 2,6-lutidine (0.175 mL, 0.16 g, 1.5 mmoles) in dichloro-methane (6 mL) was then added dropwise at −78° C. The reaction mixture was allowed to warm to room temperature, stirred for 18 h, and then washed with water (2×10 mL). The organic phase was further washed with saturated sodium chloride (10 mL), dried over anhydrous magnesium sulfate and evaporated. Purification by flash chromatography on silica gel using a gradient system, hexanes to ethyl acetate, gave 1.63 g, (74%) of the product. ¹Hmr (CDCl₃, δ): 7.34 (5H, m, Ar), 5.19 (2H, s, PhCH₂), 5.14 (1H, br d, ONH), 3.75 (1H, q, 7.0 Hz, CH), 3.69 (2H, t, 6.3 Hz, CH₂γ), 3.65 (3H, s, OCH₃), 2.33 (2H, t, 7.4 Hz, CH₂α), 1.87 (2H, m, CH₂β), 1.23 (3H, d, 7.0 Hz, CHCH₃). ¹³Cmr (CDCl₃, δ): 174.11, 173.89, 135.67, 128.55, 128.28, 128.11, 73.01, 66.55, 58.88, 51.54, 30.68, 23.91, 14.75. IR (neat): 3264, 1741, 1736 cm⁻¹. Mass spectrum (CI, m/z): 296 (M+1).

Benzyl 2R-(3-Oxo-[1,2]Oxazinan-2-yl)-Propionate (Q)

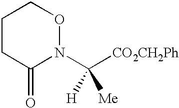

A solution of the benzyl ester (P) (1.63 g, 5.5 mmoles) in toluene (80 mL) was cooled to 0° C. and a 2.0 M solution of trimethylaluminum in toluene (5.4 mL, 10.8 mmoles) was added dropwise. The reaction mixture was allowed to warm to room temperature, stirred overnight, and then cooled to 0° C., treated dropwise with methanol (16 mL) and stirred for 20 min. Water (10 mL) was added, the ice-bath was removed and stirring was continued for 15 min. The solution was concentrated to near-dryness, 1:4 dichloromethane:tetrahydrofuran (200 mL) was added and the mixture was filtered through Celite. The filtrate was evaporated to give the crude product, 1.19 g, together with some unreacted starting material. Purification by flash chromatography on silica gel using an ethyl acetate-hexanes gradient solvent system, 10% to 40%, gave a clear oil (0.99 g, 84%). ¹Hmr (CDCl₃, δ): 7.35 (5H, m, Ar), 5.24 (1H, q, 7.3 Hz, CH), 5.20 (1H, d, 13.0 Hz, PhCH), 5.14 (1H, d, 13.0 Hz, PhCH), 4.14 (1H, m, CHγ), 3.98 (1H, m, CHγ), 2.51 (2H, m, CH₂α), 2.08 (2H, m, CH₂β), 1.50 (3H, d, 7.3 Hz, CHCH₃). ¹³Cmr (CDCl₃, δ): 170.90, 170.13, 135.45, 128.53, 128.30, 128.07, 69.32, 67.09, 53.17, 28.32, 22.11, 13.76. IR (neat): 1744, 1660 cm⁻¹. Mass spectrum (CI, m/z): 264 (M+1).

2R-(3-Oxo-[1,2]Oxazinan-2-yl)-Propionic Acid (R)

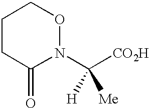

To ethyl acetate (15 mL) was added 10% palladium on activated carbon (83.0 mg, 0.078 mmole) and the suspension was stirred in an atmosphere of hydrogen for 1 h. The benzyl ester (Q) (0.206 g, 0.78 mmole) in ethyl acetate (15 mL) was then added and stirring was continued for 24 h. The mixture was filtered through Celite, which was washed with ethyl acetate (2×4 mL), and the combined filtrates were evaporated to give a white solid (0.13 g, 99%). ¹Hmr (CDCl₃, δ): 5.22 (1H, q, 7.3 Hz, CH), 4.23 (1H, m, CHγ), 4.05 (1H, m, CHγ), 2.56 (2H, m, CH₂α), 2.13 (2H, m, CH₂β), 1.52 (3H, d, 7.3 Hz, CHCH₃). ¹³Cmr (CDCl₃, δ): 173.78, 171.18, 69.61, 53.19, 28.26, 22.09, 13.58. IR (KBr): 1744, 1621 cm⁻¹. Mass spectrum (CI, m/z): 174 (M+1). Calcd. for C₇H₁₁NO₄: C, 49.54; H, 6.61; N, 8.09. Found: C, 49.36; H, 6.52; N, 7.86.

### Example 4

### Synthesis of 2R-(3-Oxo-4-Phenylacetylamino-[1,2]Oxazinan-2-yl)-Propionic Acid

Compound S

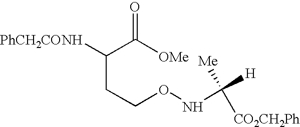

A solution of 2,6-lutidine (0.60 mL, 0.54 g 5.0 mmoles) in dichloromethane (3 mL) was added, with stirring, to a cooled (−78° C.) solution of benzyl (S)-lactate (0.450 g, 2.5 mmoles) in dichloromethane (15 mL). After 10 min, trifluoromethanesulfonic anhydride (0.42 mL, 0.71 g, 2.5 mmoles) was added dropwise and stirring was continued at −78 to −50° C. for an additional 30 min. A solution of methyl 2-phenylacetylamino-4-aminooxybutyrate (E) (0.678 g, 2.5 mmoles) and 2,6-lutidine (0.30 mL, 0.27 g, 2.5 mmoles) in dichloromethane (15 mL) was then added dropwise. When the addition was complete, the reaction mixture was allowed to warm to room temperature and stirred for 18 h. Water (2×10 mL) was added and the separated organic phase was washed with saturated sodium chloride (10 mL), dried over anhydrous magnesium sulfate and evaporated. The residue was purified by flash chromatography on silica gel using a gradient system, hexanes to ethyl acetate, to give an oil (0.304 g, 29%). 4-Phenylacetylamino-[1,2]oxazinan-3-one was also found as a side product (0.136 g, 23%). ¹Hmr (CDCl₃, δ): 7.34 (10H, m, PhCH₂CO, PhCH₂O, both isomers), 6.35 (1H, d, 7.9 Hz, CONH, one isomer, exchanges in CD₃OD), 6.32 (1H, d, 7.5 Hz, CONH, second isomer, exchanges in CD₃OD), 5.77 (1H, d, 9.5 Hz, ONH, one isomer, exchanges in CD₃OD), 5.74 (1H, d, 8.9 Hz, ONH, second isomer, exchanges in CD₃OD), 5.16 (2H, d, 4.12 Hz, PhCH₂O, one isomer), 5.15 (2H, d, 4.12 Hz, PhCH₂O, second isomer), 4.62 (1H, m, CHα, both isomers), 3.69 (2H, t, 6.3 Hz, CH₂γ, both isomers), 3.68 (3H, s, OCH₃, one isomer), 3.67 (3H, s, OCH₃, second isomer), 3.68-3.66 (1H, m, CHCH₃, both isomers), 3.59 (2H, m, PhCH₂CO, both isomers), 2.01 (2H, m, CH₂β, both isomers), 1.19 (3H, d, 7.2 Hz, CHCH₃, one isomer) 1.18 (3H, d, 7.2 Hz, CHCH₃, second isomer). ¹³Cmr (CDCl₃, δ):173.56, 172.32, 170.82, 135.55, 134.74, 129.37, 128.90, 128.87, 128.59, 128.21, .127.25, 70.30 (PhCH₂O, one isomer), 70.09 (PhCH₂O, second isomer), 66.76 (CH₂γ, both isomers), 58.63(CHCH₃, one isomer), 58.57 (CHCH₃, second isomer), 52.36 (PhCH₂CO, both isomers), 50.23 (OCH₃, both isomers), 43.50 (CHα, both isomers), 30.45 (CH₂β, both isomers), 14.72 (CHCH₃, one isomer), 14.62 (CHCH₃, second isomer). IR (CH₂Cl₂): 3286, 1742, 1660 cm⁻¹ Mass spectrum (CI, m/z): 429 (M+1). Calcd. for C₂₃H₂₈N₂O₆.0.75H₂O: C, 62.51; H, 6.68; N, 6.34. Found: C, 62.36; H, 6.35; N, 6.38.

2-(α-tert-Butoxycarbonylethyl)-4-phenylacetylamino-1,2-oxazinan-3-one (T).

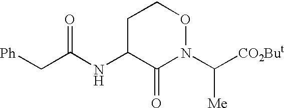

To a stirred solution of 4-phenylacetylamino-1,2-oxazinan-3-one (L) (0.447 g, 1.91 mmol) and tert-butyl (S)-α-bromopropionate (0.398 g, 1.91 mmol) in dry DMF (15 mL), KF/Al₂O₃ (40% wt, 1.94 g) was added in one portion and stirring was continued for 63 h at RT. The reaction mixture was diluted with EtOAc (40 mL) and filtered through a pad of Silica Gel 60H (for TLC) followed by washing with EtOAc (30 mL). The filtrate was washed with water (4×30 mL), 10% aqueous KHSO₄ (15 mL), sat. aq. NaHCO₃ (15 mL), brine (30 mL) and dried over MgSO₄. Removal of the solvent provided 0.621 g of a yellowish oil, which was purified by flash chromatography (Silica Gel 60, 15% EtOAc-hexanes→70% EtOAc-hexanes) to afford diastereomers TA (0.183 g) and TB (0.215 g) (57% total yield) as colorless oils.

TA: ¹H NMR (400 MHz) in CDCl₃ (J, Hz): δ1.42 (3H, d, ³J=7.4, α-Me), 1.44 (9H, CMe₃), 1.69 (1H, m, ²J=−12.1, ³J=10.0, 12.0, C5-H1), 2.91 (1H, m, ²J=−12.1, ³J=4.8, 6.9, 7.0, C5-H2), 3.60 (2H, CH₂Ph), 4.14 (1H, m, ²J=−10.1, ³J=7.0, 10.0, C6-H4), 4.40 (1H, m, ²J=−10.1, ³J=4.8, 10.0, C6-H3), 4.67 (1H, m, ³J=6.8, 6.9, 12.0, C4-H5), 4.95 (1H, q, ³J=7.4, α-CH), 6.49 (1H, br. d, ³J=6.8, NH), 7.26-7.37 (5H, m, Ph). ¹³C (100 MHz) in CDCl₃: δ14.06, 27.93, 29.24, 47.51, 54.33, 69.00, 82.30, 127.34, 128.94, 129.32, 134.51, 169.21, 170.28, 170.86. IR (film), cm⁻¹: 3319, 2988, 1737, 1658, 1537, 1370. MS (CI, isobutane), m/Z: 363 (M⁺+1).

TB: ¹H NMR (400 MHz) in CDCl₃ (J, Hz): δ1.42 (9H, CMe₃), 1.44 (3H, d, ³J=7.3, α-Me), 1.61 (1H, m, ²J=−12.4, C5-H1), 2.95 (1H, m, ²J=−12.4, ³J=4.9, 7.2 10.1, C5-H2), 3.62 (2H, CH₂Ph), 4.02 (1H, m, ²J=−10.2, ³J=4.9, 10.1, C6-H4), 4.24 (1H, m, ²J=−10.2, ³J=6.9, 10.3, C6-H3), 4.71 (1H, m, ³J=5.6, 7.2, 11.7, C4-H5), 4.87 (1H, q, ³J=7.3, α-CH), 6.48 (1H, br. d, ³J=5.6, NH), 7.24-7.37 (5H, m, Ph). ¹³C (100 MHz) in CDCl₃ δ13.40, 27.90, 28.94, 43.67, 47.77, 54.86, 69.15, 82.38, 127.36, 128.96, 129.35, 134.52, 168.27, 170.87, 171.94. IR (film), cm⁻¹: 3311, 2981, 1739, 1657, 1539, 1370. MS (CI, isobutane), m/Z: 363 (M⁺+1).

2-(α-Carboxylethyl)-4-phenylacetylamino-1,2-oxazinan-3-one (UA)

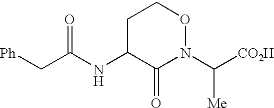

A cooled (ca. 0° C.) trifluoroacetic acid (1.5 mL) was added dropwise to tert-butyl ester TA (0.149 g, 0.41 mmol) with cooling (0˜−5° C.) and stirring. The reaction mixture was stirred for 1 h at RT and was evaporated in vacuo to dryness. The semisolid residue was triturated with dry ether and dried in vacuo to afford 0.075 g (60%) of the product UA as a white solid with mp 58-60° C. ¹H NMR (400 MHz) in CD₃OD (J, Hz): δ1.46 (3H, d, ³J=7.3, α-Me), 1.96 (1H, m, ²J=−12.5, ³J=6.5, 9.5, C5-H1), 2.56 (1H, m, ²J=−12.5, ³J=5.4, 7.4, 9.5, C5-H2), 3.61 (2H, CH₂Ph), 4.18 (1H, m, ²J=−10.3, ³J=6.5, 10.3, C6-H4), 4.43 (1H, m, ²J=−10.3, ³J=5.4, 9.5, C6-H3), 4.76 (1H, dd, ³J=7.4, 10.5, C4-H5), 5.04 (1H, q, ³J=7.3, α-CH), 7.21-7.38 (5H, m, Ph). ¹³C (100 MHz) in CD₃OD: δ14.23, 30.05, 43.61, 48.36, 55.01, 70.19, 127.90, 129.55, 130.23, 136.70, 171.89, 173.26, 174.20. IR (KBr), cm⁻¹: 3280, 2950, 1735, 1654, 1544, 1454. MS (CI, isobutane), m/Z: 307 (M⁺+1).

2-(α-Carboxylethyl)-4-phenylacetylamino-1,2-oxazinan-3-one (UB)

UB was obtained as a white solid (mp 189-190° C.) with a yield of 58% from diastereomer TB similarly to the preparation of UA (vide infra). ¹H NMR (400 MHz) in CD₃OD (J, Hz): δ1.49 (3H, d, ³J=7.3, α-Me), 1.93 (1H, m, ²J=−12.7, ³J=6.9, 9.0, 11.6, C5-H1), 2.54 (1H, m, ²J=−12.7, ³J=5.3, 7.5, 9.0, C5-H2), 3.60 (2H, CH₂PH), 4.14 (1H, m, ²J=−10.9, ³J=5.3, 9.0, C6-H4), 4.26 (1H, m, ²J=−10.9, ³J=6.9, 9.0, C6-H3), 4.77 (1H, dd, ³J=7.5, 11.6, C4-H5), 4.99 (1H, q, ³J=7.3, α-CH), 7.19-7.40 (5H, m, Ph). ¹³C (100 MHz) in CD₃OD: δ13.80, 29.98, 43.63, 48.36, 55.42, 70.44, 127.91, 129.57, 130.23, 136.66, 172.74, 172.81, 174.01. IR (KBr), cm⁻¹: 3284, 2935, 1712, 1671, 1645, 1541, 1438. MS (CI, isobutane), m/Z: 307 (M⁺+1).

### Example 5

### Synthesis of 2R-(5S-hydroxy-3-oxo-4R-phenylacetamino-[1,2]-oxazinan-2-yl)-propionic acid

Calcium L-Threonate

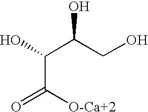

L-Ascorbic acid (52.8 g, 0.3 mol) was dissolved in water (410 mL). To the solution was added calcium carbonate (60.06 g, 0.6 mol) and the slurry was cooled to 0˜5° C. Hydrogen peroxide (50% w/w, 61.2 ml, 0.9 mol) was added dropwise at 5˜20° C. over a period of 60 minutes, and the mixture was then stirred at room temperature for sixteen hours. The reaction mixture was heated at 70˜75° C. for one hour until no more oxygen was evolved. The suspension was filtered at 70˜75° C., and the filter cake was washed with 50-60° C. hot water (30 mL×2). The combined filtrate was concentrated to about 20 mL at 55° C. under reduced pressure. Methanol (250 ml) was added dropwise to the warm concentrate (about 50° C.) until the solution became cloudy, and the mixture was then stirred at room temperature for twelve hours, filtered, and the precipitate was washed with methanol (30 mL×3). The solid was dried to a constant weight at 60° C. (3 hours) to give 45.2 grams (Yield: 94.6%) of Calcium L-threonate (Bock et al. Acta Chem. Scand. Ser. B, 37, 342-344 (1983)). IR (KBr) 1600 (s) cm⁻¹; ¹H-NMR (D₂O, 400 MHz): δ4.02 (2 1H, H_{c}, J_{bc}=2.3 Hz), 3.94 (ddd, 1H, H_{b}, J_{ab}=7.7 Hz, J_{a′b}=5.2 Hz, J_{bc}=2.2 Hz), 3.65 (ABX, 1H, H_{a′}, J_{aa′}=11.6 Hz, J_{a′b}=5.2 Hz), 3.58 (ABX, 1H, Hₐ, J_{aa′}=11.6 Hz, J_{ab}=7.7 Hz).

Methyl 2,4-dibromo-2,4-dideoxy-(1)-erythronate (MDBE)

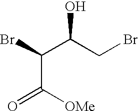

Calcium L-threonate (34.98 g, 0.11 mol) was stirred with 30% w/w HBr/HOAc solution (210 mL, 1.05 mol) for twenty four hours, and methanol (420 mL) was added. The solution was stirred at room temperature for twelve hours and the mixture was then refluxed for two hours. The solvent was evaporated to give an oil. The oil was dissolved in ethyl acetate (250 mL) and this solution was washed with brine (100 ml×1), dried over Na₂SO₄, filtered and evaporated to give 27.38 g of an oil (Yield: 90.2%) of MDBE (Wei et al. J. Org. Chem. 50, 3462-3467 (1985)). IR (film) 3340 (m), 1745 (s) cm⁻¹; ¹H-NMR (CDCl₃, 400 MHz): δ4.32 (d, 1H, H_{c}, J_{bc}=8.8 Hz), 4.24 (m, 1H, H_{b}), 3.81 (s, 3H, CO₂Me), 3.80 (AB, 2H, HₐH_{a′}); MS (CI, m/z): 277 (M+1).

Methyl 2,4-dibromo-2,4-dideoxy-3-methoxymethoxy-(L)-erythronate (CP-1)

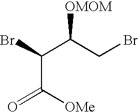

To a solution of MDBE (16.55 g, 60 mmol) in dry dichloromethane (70 ml) and dimethoxymethane (33 ml), at 0˜5° C. under nitrogen, was added BF₃-Et₂O (9.35 ml, 74 mmol) at 0˜15° C. over ten minutes. The reaction mixture was stirred at room temperature for about ten to twelve hours. The solution was cooled to about 0˜5° C. and the reaction was quenched with water (50 mL) for fifteen minutes. After extraction with dichloromethane (80 mL), the organic layer was washed with saturated NaHCO₃ (aq) (50 ml×2) and brine (100 ml×1), dried over Na₂SO₄, filtered and evaporated under reduced pressure to give a pale yellow oil 18.10 g (Yield: 92.0%) of CP-1. IR (film) 1747 (s) cm⁻¹; ¹H-NMR (CDCl₃, 400 MHz): δ4.71 (AB, 2H, H_{d}, MeOCH₂O—), 4.43 (d, 1H, H_{c}, J_{bc}=9.1 Hz), 4.16 (dt, 1H, H_{b}, J_{ab}˜J_{a′b}=2.9 Hz, J_{bc}=9.1 Hz, 3.86 (ABX, 2H, HₐH_{a′}, J_{aa′}=11.5 Hz, J_{ab}=2.8 Hz, J_{a′b}=3.1 Hz), 3.79 (s, 3H, CO₂Me), 3.41 (s, 3H, MeOCH₂O—).

Methyl 2-azido-4-bromo-2,4-dideoxy-3-methoxymethoxy-(L)-threonate

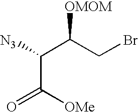

To a solution of methyl 2,4-dibromo-2,4-dideoxy-3-methoxymethoxy-(L)-erythronate (CP-1) (10.23 g, 32.0 mmol) in DMF (40 mL) was added sodium azide (NaN₃) (2.18 g, 33.5 mmol) in one portion at 10˜15° C. The mixture was stirred at 10° C.˜room temperature for twelve hours. The solution was quenched with water (35 mL) at 0˜5° C., and then extracted with ethyl acetate (80 mL×2). The combined organic solution was washed with water (20 mL×3), brine (20 mL×1), dried over Na₂SO₄, filtered and evaporated under reduced pressure to give an oil which was purified by flash chromatography using 5% ethyl acetate to afford 6.40 g (Yield 71%) of methyl 2-azido-4-bromo-2,4-dideoxy-3-methoxymethoxy-(L)-threonate. IR (film) 2115 (s), 1744 (s) cm⁻¹; ¹H-NMR (CDCl₃, 400 MHz): δ4.67 (AB, 2H, MeOCH₂O—), 4.33 (ddd, 1H, H_{b}, J_{ab}=9.0 Hz, J_{a′b}=5.0 Hz, J_{bc}=2.5 Hz), 4.29 (d, 1H, H_{c}, J_{bc}=2.5 Hz), 3.85 (s, 3H, CO₂Me), 3.58 (ABX, 1H, H_{a′}, J_{aa′}=10.2 Hz, J_{a′b}=5.0 Hz), 3.53 (ABX, 1H, Hₐ, J_{aa′}=10.2 Hz, J_{ab}=9.0 Hz), 3.36 (s, 3H, MeOCH₂O—); MS (CI, m/z), 282 (M+1), 284 (M+3).

Methyl 4-bromo-2-N-(t-butoxycarbonyl)-amino-2,4-dideoxy-3-methoxymethoxy-(L)-threonate

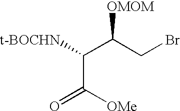

A suspension of 5% Pd/C (0.60 g) in ethyl acetate (55 mL) was activated under an atmosphere of hydrogen for two hours. Then methyl 2-azido-4-bromo-2,4-dideoxy-3-methoxymethoxy-(L)-threonate (4.30 g, 15.0 mmol) and di-t-butyl carbonate (Boc₂O) (4.00 g, 18.0 mmol) in ethyl acetate was added, and then the mixture was stirred under a hydrogen atmosphere until the starting material disappeared as monitored by ¹H-NMR (˜48 hours). The mixture was filtered through Celite and the Celite pad was washed with ethyl acetate (10 ml×3) The filtrate was concentrated under reduced pressure to give the crude product which was purified by flash chromatography (12% ethyl acetate in hexane) to isolate 4.03 g (Yield: 74.0%) of methyl 4-bromo-2-N-(t-butoxycarbonyl)-amino-2,4-dideoxy-3-methoxymethoxy-(L)-threonate as a colorless oil. IR (film) 1754 (s), 1721 (s) cm⁻¹; ¹H-NMR (CDCl₃, 400 MHz): δ5.19 (d, 1H, NH, J_{NHHc}=10.0 Hz), 4.72 (d, 1H, H_{c}, J_{NHHc}=10.3 Hz), 4.64 (AB, 2H, MeOCH₂O—), 4.36 (t, 1H, H_{b}, J_{ab}=6.4), 3.77 (s, 3H, CO₂Me), 3.43 (ABX, 2H, Hₐ, J_{ab}=6.4 Hz), 3.31 (s, 3H, MeOCH₂O—), 1.48 (s, 9H, —CO₂CMe₃); MS (CI, m/z), 356 (M+1), 358 (M+3).

4-O-[Methyl-2-N-(t-butoxycarbonyl)-amino-2-deoxy-3-methoxymethoxy-(L)-threonate]-N-hydroxyphthalimide

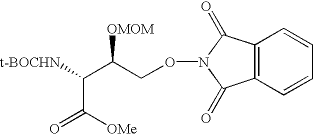

To a solution of methyl 4-bromo-2-N-(t-butoxycarbonyl)-amino-2,4-dideoxy-3-methoxymethoxy-(L)-threonate (4.40 g, 12.4 mmol) in DMF (10 mL), N-hydroxyphthalimide (2.40 g, 14.8 mmol) and DBU (2.20 g, 14.4 mmol) in DMF (25 ml) solution were added drop wise at 10˜15° C. The mixture was stirred at 10° C. to about room temperature for sixty hours. The solution was diluted with ethyl acetate (120 mL), washed with saturated NaHCO₃ (aq) (15 ml×3), brine (20 ml×1), dried over Na₂SO₄, filtered and evaporated to give an oil which was purified by flash chromatography using 30% ethyl acetate in hexane to afford 2.93 g (Yield: 54.0%) of 4-O-[methyl-2-N-(t-butoxycarbonyl)-amino-2-deoxy-3-methoxymethoxy-(L)-threonate]-N-hydroxyphthalimide as a colorless oil. IR (film) 1739 (s), 1732 (s), 1719 (s) cm⁻¹; ¹H-NMR (CDCl₃, 400 MHz): δ7.80 (AB, 4H), 5.28 (d, 1H, NH, J_{NHHc}=9.8 Hz), 4.76 (AB, 2H, MeOCH₂O—), 4.65 (d, 1H, H_{c}, J_{NHHc}=9.8 Hz), 4.58 (m, 1H, H_{b}, J_{ab}=7.0 Hz, J_{a′b}=4.8 Hz, J_{bc}=1.5 Hz); 4.36 (ABX, 1H, H_{a′}, J_{aa′}=10.6 Hz, J_{a′b}=4.8 Hz), 4.28 (ABX, 1H, Hₐ, J_{aa′}=10.6 Hz, J_{ab}=7.0 Hz), 3.79 (s, 3H, CO₂Me), 3.31 (s, 3H, MeOCH₂O—), 1.43 (s, 9H, —CO₂CMe₃); MS (CI, m/z), 439 (M+1).

Methyl 4-aminooxy-2-N-(t-butoxycarbonyl)-amino-2-deoxy-3-methoxymethoxy-(L)-threonate

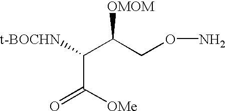

A solution of 4-O-[methyl-2-N-(t-butoxycarbonyl)-amino-2-deoxy-3-methoxymethoxy-(L)-threonate]-N-hydroxyphthalimide (2.40 g, 5.48 mmol) in dichloromethane (25 ml) was treated with methylhydrazine (0.38 g, 8.26 mmol) in dichloromethane (2 ml) solution at −15˜10° C., and then stirred at −15° C. to about room temperature for four hours. The solvent and excess methyl hydrazine were evaporated under reduced pressure. The concentrate was triturated with ethyl acetate (3 mL×6), and filtered through short path silica gel. The filtrate was evaporated to afford 1.44 g (Yield 85.3%) of methyl 4-aminooxy-2-N-(t-butoxycarbonyl)-amino-2-deoxy-3-methoxymethoxy-(L)-threonate as a colorless oil. IR (film) 3451 (b), 3323 (m), 1745 (s), 1714 (s) cm⁻¹; ¹H-NMR (CDCl₃, 400 MHz): δ5.54 (b, 2H, —ONH₂), 5.25 (d, 1H, NH, J_{NHHc}=9.6 Hz), 4.61 (AB, 2H, MeOCH₂O—), 4.47 (dd, 1H, H_{c}, J_{bc}=1.6 Hz, J_{NHHc}=9.8 Hz), 4.40 (td, 1H, H_{b}, J_{ab}˜J_{a′b}=6.2 Hz, J_{bc}=1.6 Hz); 3.76 (s, 3H, CO₂Me), 3.73 (ABX, 2H, H_{aa′}, J_{aa′}=11.2 Hz, J_{a′b}=J_{ab} 6.2 Hz), 3.31 (s, 3H, MeOCH₂O—), 1.47 (s, 9H, —CO₂CMe₃); MS (CI, m/z), 309 (M+1).

Methyl 4-N-(D)-[1-benzyloxycarbonyl-1-ethanyl]-aminooxy-2-N-(t-butoxycarbonyl)-amino-2-deoxy-3-methoxymethoxy-(L)-threonate

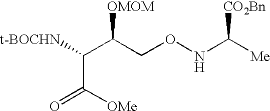

Trifluoromethanesulfonic anhydride [(CF₃SO₂)₂O] (0.79 ml, 4.69 mmol) was added dropwise to a mixture of benzyl (L)-lactate (0.84 g, 4.66 mmol) and 2,6-lutidine (0.70 g, 6.53 mmol) in dichloromethane (35 ml) at −78° C. After 20 minutes, a mixture of methyl 4-aminooxy-2-N-(t-butoxycarbonyl)-amino-2-deoxy-3-methoxymethoxy-(L)-threonate (1.30 g, 4.22 mmol)and 2,6-lutidine (0.46 g, 4.29 mmol) in dichloromethane (20 mL) was added dropwise to the reaction mixture at −78° C.˜−60° C. Stirring was continued at −78° C.˜−50° C. for one hour, the cooling bath was removed and the reaction was stirred at −60° C. to room temperature for ten hours. The reaction mixture was washed with cold water (5 ml×2), and the organic layer was dried over Na₂SO₄, filtered and evaporated to give an oil which was purified by flash chromatography using 25% ethyl acetate in hexane to afford 1.31 g (Yield 66%) of methyl 4-N-(D)-[1-benzyloxycarbonyl-1-ethanyl]-aminooxy-2-N-(t-butoxycarbonyl)-amino-2-deoxy-3-methoxymethoxy-(L)-threonate. IR (film) 3445 (b), 3367 (m), 1744 (s), 1719 (s) cm⁻¹; ¹H-NMR (CDCl₃, 400 MHz): δ7.37 (m, 5H, PhCH₂O—), 6.07 (d, 1H, —ONH, J_{ONHHd}=8.8 Hz), 5.23 (d, 1H, NH, J_{NHHc}=9.9 Hz), 5.18 (AB, 2H, PhCH₂O—), 4.56 (AB, 2H, MeOCH₂O—), 4.43 (d, 1H, H_{c}, J_{NHHc}=9.9 Hz), 4.32 (t, 1H, H_{b}, J_{ab}=6.0 Hz); 3.80 (m, 1H, H_{d}, J_{de}=7.2 Hz, J_{ONHHd}=8.8 Hz); 3.77 (d, 2H, Hₐ, J_{ab}=6.4 Hz); 3.75 (s, 3H, CO₂Me), 3.27 (s, 3H, MeOCH₂O—), 1.45 (s, 9H, —CO₂CMe₃), 1.24 (d, 3H, Hₑ, J_{de}=7.2 Hz); MS (CI, m/z), 471 (M+1).

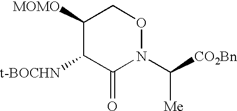

To a solution of methyl 4-N-(D)-[1-benzyloxycarbonyl-1-ethanyl]-aminooxy-2-N-(t-butoxycarbonyl)-amino-2-deoxy-3-methoxymethoxy-(L)-threonate (0.76 g, 1.62 mmol) in dry toluene (70 ml) was added dropwise 2.0 M trimethylaluminum in hexane solution (3.40 ml, 6.82 mmol) at 0˜5° C., and, after the addition, the temperature was maintained at 0˜5° C. for forty minutes, and then stirred at 5° C. to about room temperature for 3.5 hours. The reaction mixture was quenched with water at 0˜5° C., stirred at 5° C. to about room temperature for twenty minutes, and the solvent was evaporated off under reduced pressure. The concentrate was triturated with THF (5 ml×6) and dichloromethane (5 ml×4) and filtered through a silica gel/Celite pad. The filtrate was evaporated to give an oil which was purified by flash chromatography using ethyl acetate/hexane (20/80) to afford 0.45 g (Yield 62.6% ) of the product. IR (film) 3351 (w), 1745 (s), 1719 (s), 1687 (s) cm⁻¹; ¹H-NMR CDCl₃, 400 MHz): δ7.37 (m, 5H, PhCH₂OCO—), 5.16 (AB, 2H, PhCH₂O—), 5.13 (q, 1H, H_{d}, J_{de}=7.3 Hz); 4.71 (m, 1H, H_{c}), 4.64 (AB, 2H, MeOCH₂O—), 4.54 (ABM, 1H, H_{a′}, J_{aa′}=11.9 Hz, J_{a′b}=6.6 Hz); 4.00 (ABX, 1H, Hₐ, J_{aa′}=11.9 Hz, J_{ab}=3.1 Hz), 3.86 (ddd, 1H, H_{b}, Jhd ab=3.1 Hz, J_{a′b}=6.6 Hz, J_{bc}=8.5 Hz); 3.34 (s, 3H, MeOCH₂O—); 1.50 (d, 3H, Hₑ, J_{de}=7.3 Hz); 1.45 (s, 9H, —CO₂CMe₃), MS (CI, m/z), 439 (M+1).

(4D)-N-(t-butoxycarbonyl)-amino-(5L)-methoxymethoxy-1,2-oxazin-3-one

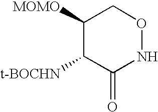

To a solution of methyl 4-aminooxy-2-N-(t-butoxycarbonyl)-amino-2-deoxy-3-methoxymethoxy-(L)-threonate (0.86 g, 2.80 mmol) in dry THF (70 ml) was added dropwise 2.0 M trimethylaluminum in hexane solution (1.68 ml, 3.86 mmol) at 0˜5° C., and, after the addition, the temperature was maintained at 0˜5° C. for forty minutes, and then stirred at 5° C. to about room temperature for 3.5 hours. The reaction mixture was quenched with water at 0˜5° C., stirred at 5° C. to about room temperature for twenty minutes, and the solvent was evaporated off under reduced pressure. The concentrate was triturated with THF (5 ml×6) and dichloromethane (5 ml×4) and filtered through a silica gel/celite pad. The filtrate was evaporated to give 0.70 g of an oil (Yield 90.0%), (4D)-N-(t-butoxycarbonyl)-amino-(5L)-methoxymethoxy-1,2-oxazin-3-one. IR (film) 3342 (m), 3232 (m), 1698 (vs) cm⁻¹; ¹H-NMR (CDCl₃, 400 MHz): δ8.80 (s, 1H, NHₑ); 5.19 (d, 1H, NH_{d}, J_{NHdHc}=7.0 Hz); 4.69 (AB, 2H, MeOCH₂O—, J=7.0 Hz), 4.57 (dd, 1H, H_{c}, J_{NHdHc}=7.1 Hz, J_{bc}=7.1 Hz); 4.26 (ABM, 1H, H_{a′}, J_{aa′}=12.2 Hz, J_{a′b}=5.9 Hz); 4.14 (ABM, 1H, Hₐ, J_{aa′}=12.2 Hz, J_{ab}=3.3 Hz); 4.26 (ddd, 1H, H_{b}, J_{ab}=3.3 Hz, J_{a′b}=5.9 Hz, J_{bc}=7.8 Hz); 3.37 (s, 3H, MeOCH₂O—); 1.45 (s, 9H, —CO₂CMe₃); MS (CI, m/z), 277 (M+1).

5-Hydroxy-4-amino-2-N-alanyl-1,2-oxazin-3-one, TFA Salt

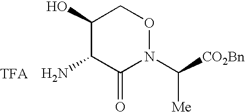

To a solution of (4D)-N-(t-butoxycarbonyl)-amino-(5L)-methoxymethoxy-2-N-(D)-[1-benzyloxycarbonylethyl]-1,2-oxazin-3-one (0.25 g, 0.57 mmol) in dichloromethane (1.0 ml) and anisole (0.5 ml) was added dropwise trifluoroacetic acid (TFA) (1.25 ml, 16.30 mmol) at 0˜5° C., and then stirred at 5° C. to room temperature for ten hours. The reaction mixture was evaporated under reduced pressure and the residue was washed with dry ether (3 ml×2) to give a solid. The solid was dried in vacuo to afford an off white powder 0.070 g (Yield: 30%) of 5-hydroxy-4-amino-2-alanyl-1,2-oxazin-3-one TFA salt. IR (KBr) 3459 (b), 3217 (m), 1731 (s), 1683 (vs) cm⁻¹; ¹H-NMR (CD₃OD, 400 MHz): δ7.36 (m, 5H, Ph); 5.18 (q, 1H, H_{d}, J_{de}=7.3 Hz); 5.17 (AB, 2H, PhCH₂O—), 4.70 (ddd, 1H, H_{b}, J_{ab}=2.6 Hz, J_{a′b}=7.1 Hz, J_{bc}=4.3 Hz); 4.58 (ABM, 1H, H_{a′}, J_{aa′}=11.8 Hz, J_{a′b}=7.1 Hz); 4.27 (d, 1H, H_{c}, J_{bc}=4.3 Hz); 3.98 (ABX, 1H, Hₐ, J_{aa′}=11.8 Hz, J_{ab}=2.6 Hz); 1.54 (d, 3H, Hₑ, J_{de}=7.3 Hz); MS (CI, m/z), 295 (M+1).

(4D)-N-(Phenylacetyl)-amino-(5L)-hydroxy-2-N-(D)-[1-benzyloxycarbonylethyl]-1,2-oxazin-3-one

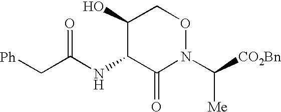

To a solution of 5-hydroxy-4-amino-2,N-alanyl-1,2-oxazin-3-one TFA salt (70 mg, 0.17 mmol) in dichloromethane was added dropwise triethylamine (52.0 mg, 0.51 mmol) at −20˜−10° C., followed by a solution of phenylacetyl chloride (31.7 mg, 0.20 mmol) in dichloromethane (1.0 ml), and then stirred at −20° C. to room temperature for six hours. The reaction mixture was diluted with dichloromethane (10 ml), washed successively with 5° C. water (1.5 ml×1), saturated NaHCO₃ (aq) (1.5 ml×3) and saturated brine (1.5 ml×1), dried over sodium sulfate, filtered and evaporated to give an oil which was purified by flash chromatography using ethyl acetate as eluent to afford an off white powder 37.8 mg (Yield 54%) of (4D)-N-(phenylacetyl)-amino-(5L)-hydroxy-2-N-(D)-[1-benzyloxycarbonylethyl]-1,2-oxazin-3-one. IR (KBr) 3464 (b), 3304 (m), 1736 (s), 1681 (s), 1644 (vs) cm⁻¹; ¹H-NMR (CD₃CN, 400 MHz): δ7.37 (m, 10H, Ph); 6.71 (d, 1H, NH, J_{NHHc}=7.4 Hz); 5.11 (AB, 2H, PhCH₂O—), 5.07 (q, 1H, H_{d}, J_{de}=7.2 Hz); 4.67 (dd, 1H, H_{c}, J_{bc}=3.8 Hz, J_{NHHc}=7.4 Hz); 4.55 (m, 1H, H_{b}); 4.48 (ABM, 1H, H_{a′}, J_{aa′}=11.6 Hz, J_{a′b}=7.4 Hz); 3.86 (ABX, 1H, Hₐ, J_{aa′}=11.6 Hz, J_{ab}=2.4 Hz); 3.65 (d, 1H, OH, J_{OHHb}=4.4 Hz), 3.61 (AB, 2H, PhCH₂CONH—), 1.45 (d, 3H, Hₑ, J_{de}=7.2 Hz); MS (CI, m/z), 413 (M+1).

(4D)-N-Phenylacetyl)-amino-(5L)-hydroxy-2-N-(D)-alanyl-1,2-oxazin-3-one

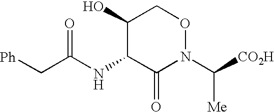

A suspension of 5% Pd/C (0.035 g) in ethyl acetate was activated under a hydrogen atmosphere for two hours. Then, (4D)-N-(phenylacetyl)-amino-(5L)-hydroxy-2-N-(D)-[1-benzyloxycarbonylethyl]-1,2-oxazin-3-one (0.035 g, 0.085 mmol) in ethyl acetate (10 ml) was added, and the mixture was stirred under a hydrogen atmosphere until starting material had disappeared as monitored by ¹H-NMR (˜4 hrs). The mixture was filtered through Celite and the Celite pad was washed with methanol (5 ml×4). The filtrate was concentrated under reduced pressure to give 0.025 g of a white powder (Yield 92.0%) of (4D)-N-phenylacetyl)-amino-(5L)-hydroxy-2-N-(D)-alanyl-1,2-oxazin-3-one. IR (KBr) 3459 (b), 3283 (w), 1727 (w), 1666 (s), 1644 (vs) cm⁻¹; ¹H-NMR (CD₃OD, 400 MHz): δ7.30 (m, 5H, Ph); 4.97 (q, 1H, H_{d}, J_{de}=7.3 Hz); 4.80 (m, 1H, H_{c}); 4.57 (m, 1H, H_{b}, J_{ab}=1.8 Hz, J_{a′b}=7.4 Hz, J_{bc}=3.7 Hz); 4.52 (ABM, 1H, H_{a′}, J_{aa′}=10.9 Hz, J_{a′b}=7.4 Hz); 3.92 (ABX, 1H, Hₐ, J_{aa′}=10.9 Hz, J_{ab}=1.8 Hz); 3.68 (AB, 2H, PhCH₂ONH—), 1.50 (d, 3H, Hₑ, J_{de}=7.3 Hz); MS (CI, m/z), 323 (M+1).

### Example 6

### Method of Determining Antibiotic Activity of Oxazinone Compounds

Materials

Agar Medium (1%w/v): Yeast extract (1.5 g), peptone (2.5 g), glucose (0.5 g) and agar (5.0 g) were dissolved in deionized water, the volume was diluted to 500 mL and the solution was sterilized by autoclaving at 121° C. for 20 min.

Preparation of Agar Plates: Under sterile conditions, the agar medium (150 mL) was heated to 100° C., cooled to 50° C. and distributed into ten petri dishes. Once the medium had hardened, the plates were inverted and stored at 4° C.

Liquid Medium: Yeast extract (0.3 g), peptone (0.5 g) and glucose (0.1 g) were dissolved in deionized water, the volume was diluted to 100 mL and the solution was sterilized by autoclaving at 121° C. for 20 min.

Methods

Under sterile conditions, freeze-dried Micrococcus luteus was dissolved in liquid medium (10 mL) and incubated at 250 rpm for 12 hours at 30° C. Liquid bacterial culture (0.5 mL) and 40% glycerol in deionized water (0.5 mL) were vortexed under sterile conditions, in a tube fitted with a screw cap and an air-tight gasket. The resulting 20% glycerol bacterial stock solution was stored at −80° C. An inoculating needle was dipped into 20% glycerol bacterial stock solution and, under sterile conditions, streaked onto the surface of an agar plate. The inverted plate was incubated at 30° C. for 12 hours and then stored at 4° C.

Under sterile conditions, a bacterial colony of Micrococcus luteus was lifted from the surface of the agar plate using a toothpick, added to liquid medium (10 mL), and incubated at 250 rpm for 12 hours at 30° C. Under sterile conditions, the agar medium (150 mL) was heated to 100° C., cooled to 50° C., liquid bacterial culture (4 mL) was added with swirling, and the medium was distributed into ten petri dishes. Once the medium had hardened, the plates were inverted and stored at 4° C.

A sterile filter disc was treated with an aliquot of a known concentration of the test compound and the solvent was removed by air drying under sterile conditions. This disc, a solvent control disc and a disc containing a known weight of a penicillin or cephalosporin, were placed on an agar plate seeded with Micrococcus luteus. The plate was inverted and incubated for 12 hours at 30° C.

The products of the synthetic methodology described in Examples 1-5 were tested on agar plates for antibacterial activity against Micrococcus luteus. DL-Cyclocanaline and Compound R exhibited activity against this particular organism as shown in Table 1.

TABLE 1

| Compounds | Weight (micrograms) | Zone size (mm) |
|---|---|---|
| solvent control | 10 | 0 |
| desacetoxycephalosporin G | 10 | 39 |
| DL-cyclocanaline | 600 | 58 |
| DL-cyclocanaline | 400 | 48 |
| R | 400 | 23 |

Equivalents

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference.

1. A method for synthesizing an oxazinone compound, comprising contacting an aminooxy compound with a cyclizing agent under appropriate conditions, such that an oxazinone compound or an acceptable salt is formed, wherein said aminooxy compound is of formula (II): 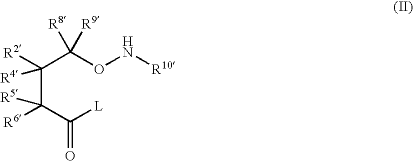
wherein:
L is a leaving group;
R^{2′}, R^{4′}, R^{5′}, R^{6′}, R^{8′} and R^{9′} are each independently selected substituting moieties; and
R^{10′} is hydrogen or an amino acid mimicking group; and wherein said oxazinone compound is of formula (I): 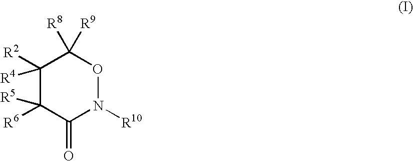
wherein
R², R⁴, R⁵, R⁶, R⁸ and R⁹ are each independently selected substituting moieties; and
R¹⁰ is hydrogen or an amino acid mimicking group.

2. The method of claim 1, wherein R⁸, R⁹, R^{8′}, and R^{9′} are each hydrogen.

3. The method of claim 1, wherein R⁴ and R^{4′} are each lower alkyl or hydrogen.

4. The method of claim 1, wherein R⁶ and R^{6′} are each lower alkyl or hydrogen.

5. The method of claim 4, wherein said appropriate conditions further comprise a deprotecting agent.

6. The method of claim 1, wherein R^{5′} is a protected amino group.

7. The method of claim 6, wherein R⁵ amino or NHCOR³, wherein R³ is an antibacterial substituent.

8. The method of claim 7, wherein said appropriate conditions further comprise a deprotecting agent.

9. The method of claim 7, wherein said appropriate conditions further comprise a derivatizing agent.

10. The method of claim 1, wherein R^{10′} is hydrogen.

11. The method of claim 1, wherein R¹⁰ is CHR¹CO₂R⁷, wherein R¹ is an amino acid side chain mimicking moiety; and R⁷ is hydrogen, a protecting moiety, or a prodrug moiety.

12. The method of claim 10, wherein said appropriate conditions further comprise an N-acylating agent.

13. The method of claim 12, wherein said N-acylating agent is contacted with said aminooxy compound.

14. The method of claim 12, wherein said N-acylating agent is contacted with said oxazinone compound.

15. The method of claim 12, wherein said N-acylating agent comprises Mitsonobu conditions.

16. The method of claim 12, wherein said N-acylating agent is a triflate reagent.

17. The method of claim 11, wherein R¹ is alkyl.

18. The method of claim 17, wherein R¹ is methyl, ethyl, propyl or butyl.

19. The method of claim 11, wherein R¹ is hydrogen.

20. The method of claim 11, wherein R⁷ is a protecting moiety.

21. The method of claim 1, wherein L is O—R^{11′}.

22. The method of claim 21, wherein R^{11′} is methyl.

23. The method of claim 1, wherein said cyclizing agent is AlMe₃.

24. The method of claim 1, wherein R^{2′} is a protected hydroxyl group.

25. The method of claim 1, wherein R² is a hydroxyl group.

26. The method of claim 1, wherein the yield of said oxazinone compound is about 30% or greater.

27. The method of claim 26, wherein the yield of said oxazinone compound is about 50% or greater.

28. The method of claim 27, wherein the yield of said oxazinone compound is about 60% or greater.

29. The method of claim 28, wherein the yield of said oxazinone compound is about 70% or greater.

30. The method of claim 29, wherein the yield of said oxazinone compound is about 80% or greater.

31. The method of claim 1, wherein said oxazinone compound has the following stereochemistry:

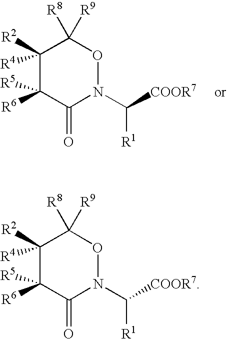

32. The method of claim 1, wherein said oxazinone compound has the following stereochemistry:

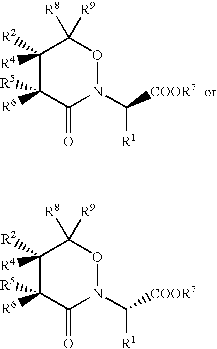

33. The method of claim 1, wherein said oxazinone compound has the following stereochemistry:

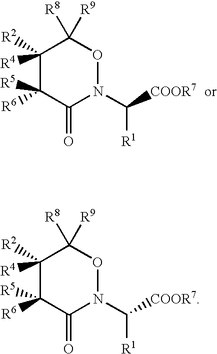

34. The method of claim 1, wherein said oxazinone compound has the following stereochemistry:

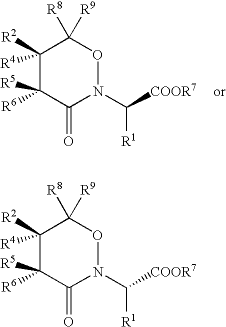

35. An aminooxy compound of the formula (II): 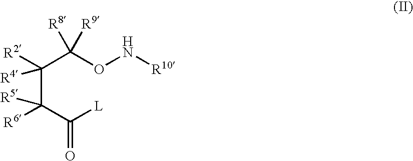
wherein:
L is a leaving group;
R^{2′}, R^{4′}, R^{5′}, R^{6′}, R^{8′} and R^{9′} are each independently selected substituting moieties; and
R^{10′} an amino acid mimicking group, or an acceptable salt thereof.

36. A method for the synthesis of an oxazinone compound, comprising contacting an compound of claim 35 under appropriate conditions, such that an oxazinone is formed.

37. A method for the synthesis of an oxazinone compound, comprising contacting an intermediate oxazinone with an N-acylating reagent under appropriate conditions, such that an oxazinone compound is formed, wherein said intermediate oxazinone is of the formula (III): 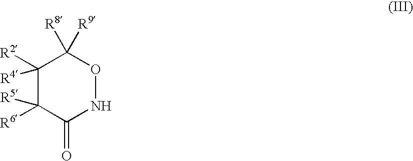
wherein:
R^{2′}, R^{4′}, R^{5′}, R^{6′}, R^{8′} and R^{9′} are each independently selected substituting moieties, and wherein said oxazinone compound is of the formula (I): 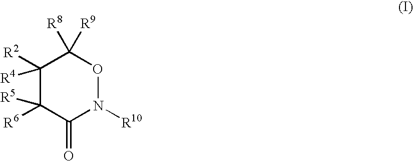
wherein:
R², R⁴, R⁵, R⁶, R⁸ and R⁹ are each independently selected substituting moieties; and
R¹⁰ is an amino acid mimicking moiety.

38. The method of claim 37, wherein said N-acylating reagent is of the formula (IV): 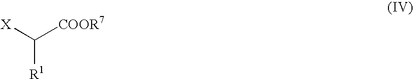
wherein
X is a halogen;
R¹ is an amino acid side chain mimicking moiety; and
R⁷ is hydrogen, a protecting moiety, or a prodrug moiety.

39. The method of claim 38, wherein X is bromine.

40. The method of claim 38, wherein said appropriate conditions comprise Al₂O₃.
